(19) United States
(12) Patent Application Publication
(10) Pub. No.: US 2022/0322960 A1
(43) Pub. Date: Oct. 13, 2022

(54) SEPARATE-ELECTRODE ELECTRIC FIELD GUIDANCE

(71) Applicant: Navix International Limited, Road Town, Tortola (VG)

(72) Inventors: Shlomo BEN-HAIM, Milan (IT); Eli DICHTERMAN, Haifa (IL)

(73) Assignee: Navix International Limited, Road Town, Tortola (VG)

(21) Appl. No.: 17/641,476

(22) PCT Filed: Sep. 11, 2020
(86) PCT No.: PCT/EP2020/075566

§ 371 Date: Mar. 9, 2022

Related U.S. Application Data
(60) Provisional application No. 62/898,581, filed on Sep. 11, 2019.

(51) Int. Cl. A61B 5/06; A61B 5/0538; A61B 5/00

(52) U.S. Cl. CPC A61B 5/065; A61B 5/0538; A61B 5/6852

(57) ABSTRACT

Electrical field-guided positioning of a second device within a body cavity, using electrical field mapping information generated from electrical field measurements by electrodes of a first device. The first device, in some embodiments, is a catheter electrode probe, and the second device is an internally implantable and/or operated medical device. An exposed, electrically conductive portion of the second device is optionally configured to be used as an electrical field measuring electrode. A rule is applied to measurements made by this electrode to estimate its position within a body cavity. The rule is generated, in some embodiments, using measurements made by the first device.

### FIELD AND BACKGROUND OF THE INVENTION

The present invention, in some embodiments thereof, relates to the field of navigation within body cavities by intrabody devices, and more particularly, to guidance of the placement of intrabody devices, optionally including implantable devices.

Several medical procedures in cardiology and other medical fields comprise the use of intrabody devices such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques and systems such as the use of electrical field measurement-guided position sensing systems.

A variety of catheter-delivered intrabody devices are in current use for purposes of treatment and/or diagnosis, including implantable pacemakers, stents, implantable rings, implantable valve replacements (such as: aortic valve replacement, mitral valve replacement and tricuspid valve replacement), left atrial appendage (LAA) occluders, and/or atrial septal defect (ASD) occluders.

### SUMMARY OF THE INVENTION

According to a first aspect of some embodiments of the present disclosure, there is provided a method of guiding an electrically conductive medical implement inside a body cavity, the method including: accessing a rule for transforming electrical field measurements to positions in the body cavity, wherein the rule is generated based on a first set of electrical field measurements of one or more electrical fields extending through the body cavity and which: are measured using electrodes of a probe at various positions of the prove within the body cavity, the probe, the probe including a plurality of measuring electrodes; receiving a second set of electrical field measurements of the one or more electrical fields measured via an electrically conductive medical implement within the body cavity, wherein the electrically conductive medical implement is a separate device to the probe; and estimating a position of the electrically conductive medical implement when the second set of electrical field measurements was measured, using the second set of electrical field measurements and the rule.

According to some embodiments of the present disclosure, the electrically conductive medical implement includes an electrically conductive portion having a surface of at least 10 mm in each of one or more dimensions, and is configured to expose the surface of the electrically conductive portion.

According to some embodiments of the present disclosure, the electrically conductive medical implement includes an electrically conductive portion at least twice as big as each of the measuring electrodes of the probe in one or more dimensions, and is configured to expose the electrically conductive portion.

According to some embodiments, the electrically conductive medical implement is configured to deploy, thereby exposing the electrically conductive portion to the electrical fields.

According to some embodiments of the present disclosure, the second set of electrical field measurements are received from the electrically conductive medical implement via an electrically conductive member which is also a structural member operable to move the electrically conducive medical implement within the body cavity.

According to some embodiments of the present disclosure, the second set of electrical field measurements is measured while the electrically conductive portion remains partially within a sheath from which it is configured to deploy.

According to some embodiments of the present disclosure, the method further includes: receiving a third and a fourth set of electrical field measurements of the one or more electrical fields measured via the electrically conductive medical implement within the body cavity, with a second shape changed from a first shape of the electrically conductive medical implement while measuring the second set of measurements; and estimating a position of the electrically conductive medical implement when the fourth set of electrical field measurements was measured, using: the rule, the second set of electrical field measurements, the third set of electrical field measurements, and an estimated difference between positions of the electrically conductive medical implement in the first shape and in the second shape when the second set of measurements and the third set of measurements were measured, respectively.

According to some embodiments, the second set of electrical field measurements were made when the electrically conductive medical implement was configured in a first shape, and the method further comprises: receiving a third set of electrical field measurements of the one or more electrical fields, the third set measured via the electrically conductive medical implement when configured in a second shape different from the first shape; and generating a modified rule for transforming electrical field measurements made by the medical implement when configured in the second shape, to positions in the body cavity, using: the rule; the second set of electrical field measurements; the third set of electrical field measurements; and an estimated difference between positions of the electrically conductive medical when the second and third sets of electrical field measurement were measured.

According to some embodiments, the method further includes receiving a fourth set of electrical field measurements of the one or more electrical fields, the fourth set measured via the electrically conductive medical implement when configured in the second shape and positioned at a different position within the body cavity relative to the position when the third set of electrical field measurements were measured; and estimating a position of the electrically conductive medical implement when the fourth set of electrical field measurements was measured, using the fourth set of electrical field measurements and the modified rule

According to some embodiments of the present disclosure, the first shape of the electrically conductive medical implement electrically mimics the measuring characteristics of at least one of the plurality of measuring electrodes. In other words, the electrically conductive medical implement provides measuring characteristics that are similar to the measuring characteristics of at least one of the plurality of measuring electrodes of the probe.

According to some embodiments of the present disclosure, the rule for transforming electrical field measurements to positions transforms electrical field readings of the first set of electrical field measurements to positions of electrodes of the multi-electrode probe.

According to some embodiments of the present disclosure, the estimating includes applying the rule to measurements of the second set of electrical field measurements to produce a position result.

According to some embodiments of the present disclosure, the position result provides the estimated position of the electrically conductive medical implement.

According to some embodiments of the present disclosure, the method includes transforming the position result to an adjusted position result according to a model of how the electrically conductive medical implement interacts with the one or more electrical fields.

According to some embodiments of the present disclosure, the method includes transforming the position result to an adjusted position result according to a model of the shape and size of the electrically conductive medical implement.

According to some embodiments of the present disclosure, the estimating includes applying the transformed rule to produce a position result.

According to some embodiments of the present disclosure, the position result provides the estimated position of the electrically conductive medical implement.

According to some embodiments of the present disclosure, the rule for transforming electrical field measurements to positions transforms electrical field measurements of the second set of electrical field measurements to positions corresponding to measurement positions of electrodes of the probe.

According to some embodiments of the present disclosure, the rule for transforming electrical field measurements to positions is generated according to a model of how the electrically conductive medical implement interacts with the one or more electrical fields. In some embodiments, the model accounts for the interaction of at least a portion of the medical implement with the one or more electrical fields.

According to some embodiments of the present disclosure, the one or more electrical fields includes at least three electrical fields, and the second set of electrical field measurements measure from a subset of the at least three electrical fields excluding at least one electrical field of the at least three electrical fields.

According to some embodiments of the present disclosure, the medical implement includes a medical implant device configured to attach to and be left in the body.

According to some embodiments of the present disclosure, the medical implement is configured to expand from a collapsed configuration upon delivery to the body cavity, and the second set of electrical field measurements are made while the medical implement is expanded. In other words, the medical implement may be configured to expand in at least one of a longitudinal and a radial direction upon delivery to the body cavity, and the second set of electrical field measurements may be made while the medical implement is expanded.

According to some embodiments of the present disclosure, the method includes removing the probe from the body cavity before receiving the second set of measurements.

According to some embodiments of the present disclosure, the method includes receiving an indication that the probe has been moved from the body cavity before the second set of measurements was received.

According to some embodiments of the present disclosure, the rule for transforming electrical field measurements to positions is generated using inter-electrode distances of the multi-electrode probe.

According to some embodiments of the present disclosure, the rule is applied to measurements made by the electrically conductive medical implement without using an inter-electrode distance from the electrically conductive medical implement to an electrode of the conductive medical implement.

According to some embodiments of the present disclosure, the estimating includes use of a calibration of the second set of electrical field measurements to the rule using position information for the electrically conductive medical implement within the body cavity which is known separately from the second set of electrical field measurements.

According to some embodiments of the present disclosure, the position information known separately includes a known position of the electrically conductive medical implement at a septum of the heart.

According to some embodiments of the present disclosure, the position information known separately includes a position at which further advancing of the conductive medical implement is prevented by contact with a wall of the body cavity.

According to some embodiments of the present disclosure, the estimating includes use of a calibration of the second set of electrical field measurements to the rule using one or more electrically measured characteristics of the environment of the electrically conductive medical implement.

According to some embodiments of the present disclosure, the one or more electrically measured characteristics of the environment comprise a change in measured impedance.

According to some embodiments of the present disclosure, the change in measured impedance is characteristic of proximity to a wall of the body cavity.

According to some embodiments of the present disclosure, the method includes moving the conductive medical implement, based on the estimating.

According to some embodiments of the present disclosure, the electrical field measurements measure a parameter including at least one of the group consisting of current, and voltage.

According to some embodiments, the method further comprises receiving a first corrective set of measurements of the one or more electrical fields measured by one or more corrective electrodes, wherein the first corrective set of measurements were taken when the one or more corrective electrodes and the medical implement were in the cavity; receiving a second corrective set of measurements of the one or more electrical fields measured by one or more corrective electrodes, wherein the second corrective set of measurements were taken when the one or more corrective electrodes were in the cavity and the medical implement was not in the cavity; determining a measurement correction based on the first and second corrective set of measurements; and estimating the position of the medical implement using the rule and the measurement correction.

According to some embodiments, estimating the position of the medical implement comprises generating a modified rule based on the measurement correction and the rule, and subsequently applying the modified rule to the measurements of the second set of electrical field measurements to produce a position result.

According to some embodiments, estimating the position of the medical implement comprises applying the measurement correction to the second set of measurements to produce a corrected second set of measurements, and subsequently applying the rule to the corrected second set of measurements to produce a position result.

According to some embodiments, the method further comprises: receiving a first corrective set of measurements of the one or more electrical fields measured by one or more corrective electrodes, wherein the first corrective set of measurements were taken when the one or more corrective electrodes and the medical implement were in the cavity; applying the rule to the first corrective set of measurements to generate a first corrective set of positions; receiving a second corrective set of measurements of the one or more electrical fields measured by one or more corrective electrodes, wherein the second corrective set of measurements were taken when the one or more corrective electrodes were in the cavity and the medical implement was not in the cavity; applying the rule to the second corrective set of measurements to generate a second corrective set of positions; determining a position correction based on the first and second corrective set of positions; and estimating the position of the medical implement using the rule and the position correction.

According to some embodiments, estimating the position of the medical implement comprises generating a modified rule based on the position correction and the rule, and subsequently applying the modified rule to the measurements of the second set of electrical field measurements to produce a position result.

According to some embodiments, estimating the position of the medical implement comprises applying the rule to the second set of electrical field measurements to generating a first set of medical implement positions, and applying the position correction to the first set of medical implement positions to produce a position result

According to a second aspect of some embodiments of the present disclosure, there is provided a system comprising one or more processors, and one or more storage media storing instructions that, when executed by the one or more processors cause the one or more processors to carry out any of the methods disclosed herein.

According to a third aspect of some embodiments of the present disclosure, there is provided one or more computer-readable storage media storing instructions that, when executed by the one or more processors cause the one or more processors to carry out the method of any of the methods disclosed herein.

According to a fourth aspect of some embodiments of the present disclosure, a system for guiding an electrically conductive medical implement inside a body cavity is provided. The system comprises one or more processors configured to access a rule for transforming electrical field measurements to positions in the body cavity, wherein the rule is generated based on a first set of electrical field measurements measured using electrodes of a multi-electrode probe when the probe was in the body cavity; receive a second set of electrical field measurements measured via the electrically conductive medical implement when the medical implement was in the body cavity, wherein the electrically conductive medical implement is a separate device to the multi-electrode probe; and estimate a position of the electrically conductive medical implement when the second set of electrical field measurements was measured, using the second set of electrical field measurements and the rule.

In some embodiments, the one or more processors are further configured to, prior to accessing the rule: access the first set of electrical field measurements; generate, using the first set of electrical field measurements, the rule for transforming electrical field measurements to positions.

In some embodiments, the second set of electrical field measurements are received from the electrically conductive medical implement via a structural member of the medical implement, the structural member being operable to move the electrically conducive medical implement within the body cavity and being electrically conductive.

In some embodiments, the second set of electrical field measurements were made when the electrically conductive medical implement was configured in a first shape, and wherein the processor is further configured to: receive a third set of electrical field measurements of the one or more electrical fields, the third set measured via the electrically conductive medical implement when configured in a second shape different from the first shape; and generate a modified rule for transforming electrical field measurements made by the medical implement when configured in the second shape, to positions in the body cavity, using: the rule; the second set of electrical field measurements; the third set of electrical field measurements; and an estimated difference between positions of the electrically conductive medical when the second and third sets of electrical field measurement were measured.

In some embodiments, the one or more processors are further configured to: receive a fourth set of electrical field measurements of the one or more electrical fields, the fourth set measured via the electrically conductive medical implement when configured in the second shape and positioned at a different position within the body cavity relative to the position when the third set of electrical field measurements were measured; and estimate a position of the electrically conductive medical implement when the fourth set of electrical field measurements was measured, using the fourth set of electrical field measurements and the modified rule.

In some embodiments, the first shape of the electrically conductive medical implement provides measuring characteristics that are similar to the measuring characteristics of at least one of the plurality of measuring electrodes of the probe.

In some embodiments, the rule for transforming electrical field measurements to positions transforms electrical field readings of the first set of electrical field measurements to positions of electrodes of the multi-electrode probe.

In some embodiments, the one or more processors are configured to estimate the position of the electrically conductive medical implement by applying the rule to measurements of the second set of electrical field measurements to produce a position result. In some embodiments, the one or more processors are configured to transform the position result to an adjusted position result according to a model of how the electrically conductive medical implement interacts with the one or more electrical fields. In some embodiments, the one or more processors are configured to transform the position result to an adjusted position result according to a model of the shape and size of the electrically conductive medical implement.

In some embodiments, the one or more processors are configured to transform the rule according to a model of how the electrically conductive medical implement interacts with the one or more electrical fields to produce a transformed rule, and wherein the estimating comprises applying the transformed rule to produce a position result.

In some embodiments, the system further comprises a user interface configured to display the position result.

In some embodiments, the rule for transforming electrical field measurements to positions transforms electrical field measurements of the second set of electrical field measurements to positions corresponding to measurement positions of electrodes of the probe. The rule for transforming electrical field measurements to positions may be generated according to a model of how the electrically conductive medical implement interacts with the one or more electrical fields.

In some embodiments, the model accounts for the interaction of at least a portion of the second device with the one or more electrical fields.

In some embodiments, the system further comprises the medical implement. The medical implement may comprise a medical implant device configured to attach to and be left in the body. Additionally are alternatively, the medical implement may be configured to expand in at least one of a longitudinal and a radial direction upon delivery to the body cavity, and the second set of electrical field measurements are made while the medical implement is expanded.

In some embodiments, the system further comprises an electrical field measurement controller configured to receive electrical signals from the medical implement and determine the second set of electrical field measurements, wherein the second set of electrical field measurements are measurements of a parameter comprising at least one of the group consisting of current and voltage.

According to a fifth aspect of some embodiments of the present disclosure, there is provided a system for guiding a medical implement comprising an electrically conductive portion inside a body cavity. The system comprises: the medical implement; an implement electrical field measurement controller; an electrically conductive structure configured to connect the implement electrical field measurement controller to an electrically conductive portion of the medical implement, and also to mechanically manipulate the medical implement; and one or more processors configured to: access a rule for transforming electrical field measurements to positions in the body cavity; receive from said implement electrical field measurement controller and via said conductive structure a first set of implement electrical field measurements measured within the body cavity using the conductive portion of the medical implement; and estimate a position of the electrically conductive medical implement when the implement electrical field measurements were measured, using the set of implement electrical field measurements and the rule.

In some embodiments, the system further comprises a multi-electrode probe; a probe electric field measurement controller; wherein the one or more processors are further configured to: receive from the probe electrical field measurement controller a set of probe electrical field measurements measured within the body cavity using the multi-electrode probe; and generate the rule for transforming electrical field measurements to positions based on the set of probe electrical field measurements.

In some embodiments, the system further comprises: an electric field generating electrodes configured to be positioned on a surface of a body; an electrical field controlled configured to generate electrical fields in the body cavity using the electrical field generating electrodes.

In some embodiments, the medical implement comprises a medical implant device configured to attach to and be left in the body.

In some embodiments, the first set of electrical field measurements were made when the medical implement was configured in a first shape, and wherein the one or more processors are further configured to receive from the implement electrical field measurement controller and via the conductive structure a second set of implement electrical field measurements measured within the body using the electrically conductive portion when the medical implement was configured in a second shape different from the first shape; and generate a modified rule for transforming electrical field measurements made by the medical implement when configured in the second shape, to positions in the body cavity, using: the rule; the first set of implement electrical field measurements; the second set of implement electrical field measurements; and an estimated difference between positions of the electrically conductive medical when the first and second sets of implement electrical field measurements were measured.

In some embodiments, the one or more processors are further configured to: receive from the implement electrical field measurement controller and via the conductive structure a third set of implement electrical field measurements measured within the body cavity using the electrically conductive portion when the medical implement was configured in the second shape and positioned at a different position within the body cavity relative to the position when the second set of implement electrical field measurements were measured; and estimate a position of the electrically to conductive medical implement when the third set of implement electrical field measurements was measured, using the third set of implement electrical field measurements and the modified rule.

In some embodiments, the first shape of the electrically conductive medical implement provides measuring characteristics that are similar to the measuring characteristics of at least one of the plurality of measuring electrodes of the probe.

In some embodiments, the rule for transforming electrical field measurements to positions transforms electrical field readings of the first set of electrical field measurements to positions of electrodes of the multi-electrode probe. In some embodiments, the one or more processors are configured to estimate the position of the electrically conductive medical implement by applying the rule to measurements of the second set of electrical field measurements to produce a position result. In some embodiments, the one or more processors are configured to transform a position result to an adjusted position result according to a model of how the electrically conductive medical implement interacts with the one or more electrical fields.

In some embodiments, the one or more processors are configured to transform the position result to an adjusted position result according to a model of the shape and size of the electrically conductive medical implement.

In some embodiments, the one or more processors are configured to transform the rule according to a model of how the electrically conductive medical implement interacts with the one or more electrical fields to produce a transformed rule, and wherein the estimating comprises applying the transformed rule to produce a position result.

In some embodiments, the rule for transforming electrical field measurements to positions transforms electrical field measurements of the second set of electrical field measurements to positions corresponding to measurement positions of electrodes of the probe.

In some embodiments, the rule for transforming electrical field measurements to positions is generated according to a model of how the electrically conductive medical implement interacts with the one or more electrical fields.

In some embodiments, the model accounts for the interaction of at least a portion of the second device with the one or more electrical fields.

In some embodiments, the medical implement is configured to expand in at least one of a longitudinal and a radial direction upon delivery to the body cavity, and the second set of electrical field measurements are made while the medical implement is expanded.

In some embodiments, the electrical field measurements are measurements of a parameter comprising at least one of the group consisting of current and voltage.

In some embodiments, the one or more processors of the system of the fourth or fifth aspect are further configured to: receive a first corrective set of measurements of the one or more electrical fields measured by one or more corrective electrodes, wherein the first corrective set of measurements were taken when the one or more corrective electrodes and the medical implement were in the cavity; receive a second corrective set of measurements of the one or more electrical fields measured by one or more corrective electrodes, wherein the second corrective set of measurements were taken when the one or more corrective electrodes were in the cavity and the medical implement was not in the cavity; determine a measurement correction based on the first and second corrective set of measurements; and estimate the position of the medical implement using the rule and the measurement correction. In some embodiments, the one or more processors are configured to estimate the position of the medical implement by generating a modified rule based on the measurement correction and the rule, and subsequently applying the modified rule to the measurements of the second set of electrical field measurements to produce a position result. Alternatively, the one or more processors are configured to estimate the position of the medical implement by applying the measurement correction to the second set of measurements to produce a corrected second set of measurements, and subsequently applying the rule to the corrected second set of measurements to produce a position result.

In some embodiments, the one or more processors of the system of the fourth or fifth aspect are further configured to: receive a first corrective set of measurements of the one or more electrical fields measured by one or more corrective electrodes, wherein the first corrective set of measurements were taken when the one or more corrective electrodes and the medical implement were in the cavity; apply the rule to the first corrective set of measurements to generate a first corrective set of positions; receive a second corrective set of measurements of the one or more electrical fields measured by one or more corrective electrodes, wherein the second corrective set of measurements were taken when the one or more corrective electrodes were in the cavity and the medical implement was not in the cavity; apply the rule to the second corrective set of measurements to generate a second corrective set of positions; determine a position correction based on the first and second corrective set of positions; and estimate the position of the medical implement using the rule and the position correction. In some embodiments, the one or more processors are configured to estimate the position of the medical implement by generating a modified rule based on the position correction and the rule, and subsequently applying the modified rule to the measurements of the second set of electrical field measurements to produce a position result. In some embodiments, the one or more processors are configured to estimate the position of the medical implement by applying the rule to the second set of electrical field measurements to generating a first set of medical implement positions, and applying the position correction to the first set of medical implement positions to produce a position result. In some embodiments, the system further comprises the one or more corrective electrodes.

According to an aspect of some embodiments of the present disclosure, there is provided a system for guiding an electrically conductive medical implement inside a body cavity, including a processor configured to: access a first set of electrical field measurements measured via electrodes of a multi-electrode probe in the body cavity; generate, using the first set of electrical field measurements, a rule for transforming electrical field measurements to positions; receive a second set of electrical field measurements measured via an electrically conductive medical implement within the body cavity; and estimate a position of the electrically conductive medical implement using the second set of electrical field measurements and the rule.

According to an aspect of some embodiments of the present disclosure, there is provided a system for navigating an electrically conductive medical implement inside a body cavity, the system including a processor configured to: access a first set of electrical field measurements measured via electrodes of a multi-electrode probe when the multi-electrode probe was in the body cavity; generate a rule for transforming electrical field measurements to positions based on the first set of electrical field measurements; receive a second set of electrical field measurements measured via an electrically conductive medical implant when the medical implant was in the body cavity; and estimate a position of the electrically conductive medical implant when the second set of electrical field measurements was measured, based on the second set of electrical field measurements and the rule for transforming electrical field measurements to positions.

According to an aspect of some embodiments of the present disclosure, there is provided a method of guiding an electrically conductive medical implement inside a body cavity, wherein the electrically conductive medical implement is disposed within an insulating sheath, the method including: moving the electrically conductive medical implement such that it is at least partially extruded it from the insulating sheath, and at least partially expanding a shape of the electrically conductive medical implement; and measuring a plurality of electrical fields using the conductive medical implement as a measuring electrode; wherein the moving is performed by force exerted through mechanical manipulation of a conductive structure, and the measuring is performed by receiving electrical signals from the conductive medical implement transmitted over the conductive structure.

According to some embodiments of the present disclosure, the moving and measuring are performed simultaneously.

According to an aspect of some embodiments of the present disclosure, there is provided a system for guiding a medical implement including an electrically conductive portion inside a body cavity, including: the medical implement; an electrical field measurement controller; an electrically conductive structure configured to connect the electrical field measurement controller to an electrically conductive portion of the medical implement, and also to mechanically manipulate the medical implement; and a processor configured to: access a rule for transforming electrical field measurements to positions, receive from the electrical field measurement controller via the conductive structure a set of implement electrical field measurements measured within the body cavity, and estimate a position of the electrically conductive medical implement using the implement set of electrical field measurements and the rule.

According to some embodiments of the present disclosure, the system further includes: a multi-electrode probe; electrical field generating electrodes; an electrical field controller, configured to generate electrical fields using the electrical field generating electrodes; at least one electrical field measurement controller configured to measure electrical field between the ground electrode and at least two of the electrodes of the multi-electrode probe, wherein the processor is further configured to: generate the rule for transforming electrical field measurements to positions based on the first set of electrical field measurements.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A is a flowchart schematically illustrating a method of navigating a device within a body cavity using electrical field measurements, according to some embodiments of the present disclosure;

FIGS. 1B-1E schematically illustrate an example of the method of FIG. 1A applied to device positioning (of a first device, and then a second device) in a body cavity (in the illustrated example, a left atrium—shown in cross-section—of a heart), according to some embodiments of the present disclosure;

FIG. 1F schematically illustrates shows an overview of left atrium in relation to a heart, according to some embodiments of the present disclosure;

FIG. 2 is a flowchart schematically illustrating a method of positioning a second device using a combination of a mimicking configuration measurements and changed-shape configuration measurements, according to some embodiments of the present disclosure;

FIG. 3 is a flowchart schematically illustrating a method of calibrating electrical field measurements made using a second device within a body cavity to electrical field measurements made using a first device within the body cavity, according to some embodiments of the present disclosure;

FIGS. 4A-4C schematically illustrate movement of an equivalent first-device electrode position of a cage-shaped second device as the second device deploys, according to some embodiments of the present disclosure;

FIGS. 5A-5C schematically illustrate movement of an equivalent first-device electrode position of an “umbrella”-shaped second device as the second device deploys, according to some embodiments of the present disclosure;

FIGS. 6A-6C schematically illustrate movement of an equivalent first-device electrode position of a bent-linear second device as the second device deploys, according to some embodiments of the present disclosure;

FIG. 7 schematically illustrates a system for navigating a device within a body cavity using electrical field measurements, according to some embodiments of the present disclosure; and

FIG. 8 schematically represents an implantable device for modifying the circumference of a heart valve, and comprising a plurality of electrically conductive fasteners used to secure implantable device to the wall of a heart left atrium, according to some embodiments of the present disclosure.

FIGS. 9A-9B illustrate a Septal Occluder device in accordance with some embodiments of the present disclosure.

FIGS. 10A-10B illustrate a Lasso® catheter in accordance with some embodiments of the present disclosure.

### DESCRIPTION OF SPECIFIC EMBODIMENTS OF THE INVENTION

The present invention, in some embodiments thereof, relates to the field of navigation within body cavities by intrabody devices, and more particularly, to guidance of the placement of intrabody devices, optionally including implantable devices.

### Overview

An aspect of some embodiments of the present disclosure relates to electrical field-guided positioning of a second device within a body cavity, using electrical field mapping information generated from electrical field measurements by electrodes of a first device. Herein, electrical field measurements may also be referred as “electrical measurements”. Reference to ‘electrical field measurements’ as used herein may refer to direct measurements of the electrical field at the position of the measurement, or may refer to measurements of parameters, such as voltage, that are indicative of the electrical field at the position of the measurement. Electrical field measurements optionally include, for example, measurements of voltage or current.

Herein, the term “first device” refers to an electrode-carrying probe (a “probe”)—for example, an electrode catheter—insertable to and navigable within a body cavity; and configured to make electrical field measurements within the body cavity. In some embodiments, the first device carries a plurality of electrodes.

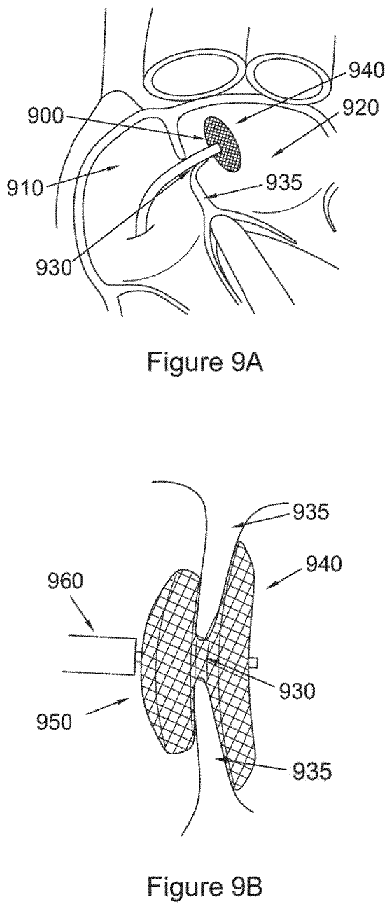

Herein, the term “second device” refers to medical devices, such as medical implements insertable to and navigable within a body cavity (e.g., to move the implement to a site of implantation and/or endolumenal operation). The medical implement (i.e., second device) may be any device insertable through a catheter, and/or another instrument such as an endoscope. Examples of medical implements (i.e., second devices) include: implantable pacemaker, stent, implantable ring, implantable valve replacement (e.g., aortic valve replacement, mitral valve replacement and/or tricuspid valve replacement), left atrial appendage (LAA) occluder, and/or atrial septal defect (ASD) occluder. In some embodiments, the second device is an Amplatzer septal occluder used to treat an atrial septal defect as described in more detail below with reference to FIGS. 9A and 9B.

In some embodiments, electrical field mapping information comprises a map which associates measurements made by the electrodes of the first device to positions of the electrodes when carrying out those measurements (that is, the measurement data are position-mapped). Using electrical field mapping to guide movements of devices within a body cavity provides potential advantages, for example by reducing or eliminating a need for use of imaging techniques which use ionizing radiation (e.g., isotope and/or X-ray imaging) and/or access-limited imaging resources (e.g., MRI).

In some embodiments, using the electrical field mapping comprises application of a rule based on the map. The rule, in some embodiments, comprises direct lookup within the map, as if the electrical field measurement made by the second device was a measurement made by the first device. In some embodiments, the rule comprises transformation of the lookup result and/or the map to match second-device measurements to the map.

A rule optionally comprises other aspects. For example, it may comprise steps of selection from among different maps and/or map transformations to suit particular conditions of the second device (e.g., different configurations, conformations, and/or degrees of unsheathing), particular conditions of electrical field generation (e.g., which fields are being generated at any given moment of a procedure), and/or particular regions of navigation (e.g., there may be different transformations of the field mapping information, each suitable for finding the position of the second device in some region of the body cavity, but not necessarily suitable for a complete and accurate transformation of all the field mapping information).

In some embodiments, the rule is defined so that the electrical fields measured using the second device are a subset of electrical fields used in measurements made by the first device (e.g., three or more electrical fields are measured during first-device mapping, but only two or three electrical fields are measured for positioning of the second device).

Herein an electrical field generated at two different times using substantially the same generating device parameters, e.g., of voltage, current, frequency, and generating electrode configuration, is considered to be practically the same electrical field. This is regardless of minor actual variations in the electrical field due to factors such as body movement and/or electrical component property drift, although these may be considered as contributing time-variability (or “noise”) to the electrical field. In some embodiments, the electrical field is continuously generated from electrodes which remain in place. However, these conditions are not strictly necessary to recreate the same electrical field parameters, and accordingly the same electrical field. For example, an electrical field generator can be briefly turned off and then on again, and the electrical field generated is still “the same” electrical field, as the concept of electrical field identity is used herein.

Furthermore, the definition herein of the identity of an electrical field over time discounts influences of measurement devices on the electrical field. To the extent that such influences occur (e.g., because the measuring device's impedance is not infinite), they are considered to modify an electrical field that still retains its “identity”.

In some embodiments, the first device comprises a sensing catheter or other device comprising 2, 3, 4, or more electrodes, and the electrical field mapping information is derived in part by using one or more known distances between the electrodes (inter-electrode distances) to set distance scaling of simultaneous electrical field measurements from each other. This scaling provides a constraint that allows estimation of relative position upon application of the constraint to a set having a suitably large number of measurements made as the first device moves about within the body cavity. The first device may itself be navigated and/or have its position determined using this estimation of relative position.

Construction and first-device use of maps of this type may be performed, for example, as described in International Patent Publication No. WO2018/130974 and/or in International Patent Publication No. WO2019/034944, the contents of which are included herein by reference in their entirety. Herein, measurement data that are position-mapped using the known inter-electrode distances are said to be “self-scaled”.

The inventors have realized that the self-scaled position-mapped measurement data (for example) provides a basis for navigation of a second device, even if the second device has only one electrode. Accordingly, in some embodiments, the method provides for navigation based on single-electrode measurements of a second device, based on self-scaled, position-mapped measurements made by a plurality of measuring electrodes of a first device—even though the second device is unsuitable for self-scaled position mapping.

This represents a potential advance, insofar as electrical field-based measurements by a single-electrode device as such are generally not susceptible to use with self-scaled position-mapped measurements (since there is only one electrode, there is no well-known inter-electrode distance).

While methods of electrical field-based guidance which do not use self-scaling are also known, their use is not always available and/or suitable. For example, these may rely on calculations of field geometry and/or careful configuration of how the electrical fields are generated, which are potentially unavailable, time-consuming, and/or prone to errors in setup and/or model assumptions.

Nevertheless, it should be understood that the electrical field mapping information used in some embodiments of the present disclosure to estimate second-device positioning is optionally obtained by a method that does not rely on self-scaling; for example, systems wherein electrical fields are generated to control their voltage gradient geometry (e.g., to be substantially linear within a certain region of interest), and/or systems wherein electrical field voltage gradients are calculable from other available information such as electrode positions and the dielectric properties and structure of body tissue through which electrical fields are generated.

Furthermore, the inventors have realized that when electrodes of both the first device and the second device are of sufficiently similar size and also of sufficiently similar impedance (e.g., pass minimal currents), any differences in how each “sees” the electrical field is potentially minimal enough that relative measurement differences made using what are essentially two different measurement systems can be ignored.

In some embodiments, an actual difference in one or more of these conditions (or another condition affecting measurement similarity) is compensated for by calibration, and/or accounted for as a limitation on the accuracy and/or precision of position estimation. For example, differences can optionally be reduced by the application of corrections which can be learned from the measurements themselves, and/or by calibration steps using equipment available at the time of the procedure, and/or pre-programmed for known devices.

For example, in some embodiments, systematic offsets and/or scale differences in the measurements can be corrected by checking for measurement differences at positions which are well-defined outside of the electrical field measurements themselves. As examples, measurements from two different devices are estimated, in some embodiments, to originate from comparable positions if they are both made by one or more of:
At a same point of insertion (such as a heart atrial septum).
While both the first and second device occupy adjacent positions, e.g., in contact with each other or at a known offset from each other.
Along a same path (for example along a same arc of a catheter as it advances into the body cavity).
At same extremes of available movement limited by lumenal walls of the body cavity.
At same landmarks such as blood vessel branching points, blood vessel ostia, heart valves, tissue folds, and/or appendages (such as the left atrial appendage).
At same electrically measured landmarks, such as local or global minima and/or maxima of measured voltage, or maximum- or minimum-amplitude slope (local or global) of voltage change; including features which include joint consideration of a plurality of voltage fields generated with different orientations, frequencies, and/or currents.
At any other “same position”, however this may be identified.

In some embodiments, calibration involves corrections to compensate for entities that influence the measurements measured by the second device. For example, whilst a conductive portion of the second device is used to measure electrical field measurements in the body cavity, the same conductive portion, as well as other, optionally dielectric portions of the second device may interact with the electrical fields in the body cavity. As would be understood by the skilled person, such conductive or dielectric portions would interact with and modify the electrical field, thereby affecting the electrical field measurements made using the electrode conductive portion of the second device. This would lead to a mapping of the second device measurements to wrong positions in the body cavity, and so a correction may be required to account for the field modifying portions (referred to herein as field modifiers). As described in more detail below correcting errors can involve one or more of: correcting the rule for mapping measurements to positions, correcting the measurements before applying the measurements to the original rule, or correcting the resulting positions provided after applying the measurements to the original rule. The original rule (that does not account for field modifiers) is referred to below as a baseline rule.

Since the second device is typically a medical implement such as an implant, the second device may have one or more field modifiers that need to be accounted for when guiding the second device in the body cavity based on the measurements made using the second device itself. The following examples are therefore of particular interest in the event that the second device comprises field modifying portions. As mentioned above, the field modifying portions may be conductive or dielectric, or may otherwise be any portion of the second device that modifies the electric field.

In some embodiments, the errors generated as a result of applying the baseline rule to the second device measurements are corrected at least in a region of interest within the body cavity. For example, in some embodiments, the second device comprising the field modifier is an implement to be implanted in a certain position within the cavity, and so it is useful to have the highest accuracy of the navigation at that certain position or at least in the vicinity of that position. In the following, the vicinity of the certain position is referred to as a region of interest, but the term region of interest may be used for regions being of interest for other reasons. In some embodiments, the errors are corrected by modifying the baseline rule, so that the modified rule provides corrected positions. In some embodiments, the baseline rule is used, and the erroneous positions it provides are corrected.

In more detail, the correction to the measurements/baseline rule/positions is determined using one or more corrective electrodes. The specific examples below refer to a single corrective electrode, but it would be appreciated that a plurality of corrective electrodes may equally be used. The corrective electrode(s) are disposed on a device (referred to herein as the corrective device) separate to the second device that is configured to be disposed in the body cavity. The corrective device may be a catheter device similar to the first device used to obtain measurements for generating the baseline rule. In some embodiments, the corrective device may be a catheter of a different kind. For example, the first device may be configured for navigate in the entire body cavity, while the corrective device may be configured to be anchored to the region of interest. The position of the corrective electrode may be restricted to the region of interest in the body cavity.

The corrective electrode may be used to obtain electrical field measurements in the region of interest in the presence of the field modifier (e.g. when the second device is also in the vicinity of the region of interest). These measurements taken using the corrective electrode in the presence of the field modifier are then used as follows to compensate for the presence of the field modifier.

In a first example, the measurements taken using the corrective electrode in the presence of the field modifier are mapped to positions by applying the baseline rule (thereby giving inaccurate positions of the corrective electrode). These positions are compared to a known position of the corrective electrode. The comparison of the inaccurate positions obtained by measurements and the known positions can be used to determine a correction. The known position of the corrective electrode can be obtained by applying the baseline rule to measurements taken using the corrective electrode, when the measurements were taken before the field modifier (e.g. the second device) was introduced to the region of interest. In other words, the known position of the corrective electrode can be obtained using measurements obtained in the absence of the field modifier. This assumes that the corrective electrode does not change position in the region of interest between the measurements taken in the absence and the presence of the field modifier. In other embodiments, the known position may be obtained using various imaging techniques as would be known to the skilled person, or any other suitable technique that is independent of the measurements made using the electrode itself.

For example, the baseline rule may be modified so as to transform readings of the corrective electrode in the presence of the field modifier to the known position of the corrective electrode. In another example, results of applying the baseline rule to readings of the corrective electrode obtained in the presence of the field modifier are modified to fall on the known position of the corrective electrode. In another example, the readings of the corrective electrode are modified such that applying the baseline rule to the modified readings results in positions corresponding to the known positions of the corrective electrode.

In a second example, measurements are taken using the corrective electrode in the absence of the field modifier (when the second device is not in the region of interest or in the vicinity of the region), and further measurements are taken using the corrective electrode in the presence of the field modifier (when the second device is also in the region on interest). The measurements taken using the corrective electrode in the presence and absence of the field modifier may be taken in any order. A corrective registration function is then defined based on these two sets of measurements. In particular, the corrective registration function may be defined based on a group of positions obtained by applying the baseline rule to the measurements in the presence of the field modifier (denoted {a}), and a group of positions obtained by applying the baseline rule to the measurements in the absence of the field modifier (denoted {b}). The corrective registration function is defined as a registration transformation that transforms each member of {a} to the corresponding member of {b}, that is, the position of each corrective electrode as provided by the baseline rule in the presence of the field modifier is registered with the position of the same corrective electrode as provided by the baseline rule in the absence of the field modifier. This ensures that the readings of the corrective electrode in the presence of the field modifier are registered to the correct positions (i.e. the positions provided by measurements in the absence of the field modifier). This condition may be symbolized as:

f({a})={b}

In addition, the corrective function may be required to register positions obtained by the baseline rule in the absence of the field modifier to themselves (i.e. the positions in the cavity that are not in the vicinity the region of interest and for which the position provided by the baseline rule is the best available approximation of their current position. These positions are denoted {X}. To facilitate smoothness of the corrective function, the latter condition may be made by requiring that the positions in {X}∪{a} are registered to the positions in {X}∪{b}. That is:

f({X}∪{a})→{X}∪{b}

Thus, once measurements are obtained using the second device in the presence of the field modifier, they may be mapped to positions by first applying the baseline rule to the readings to obtain inaccurate positions, and then applying the corrective function f to the inaccurate positions to obtain corrected positions.

In some embodiments, similar logic is applied by correcting the electrical field measurements, rather than the positions obtained from the measurements. In such embodiments, a corrective function g is generated based on measurements taken using the corrective electrode(s) in absence of the field modifier, symbolized herein by the symbol {y}; and measurements taken using the corrective electrode(s) in presence of the field modifier, symbolized herein by the symbol {z}. The corrective function g is required to be smooth, and fulfill the following two conditions:

g({z})={y};

g:({V}∪{z})→{V}∪{y}

Where {V} are the measurements taken at positions that are not in the vicinity of the region of interest.

In such embodiments, readings received from the second device are first corrected by the corrective function, and the corrected readings are transformed to corrected positions by the baseline rule.

It is noted that the field modifier is not necessarily static. For example, it may move from one place to another (for example as the second device moves), causing at each place a different field modification. Additionally or alternatively, the field modifier may change orientation (for example as the second device changes orientation), and cause at each orientation a different field modification. Additionally or alternatively, the field modifier may be electrified, so that its dielectric properties change, and it may modify the field differently even without changing position and/or orientation. Additionally or alternatively, the field modifier may change shape or configuration. For example, the field modifier may a medical implement that is deployable from an insulating sheath, and the extent of modification of the electrical field may change depending on whether the field modifier is in a deployed configuration, or if the field modifier is retracted inside the sheath. In all such cases, the above procedures may be repeated so as to allow following the field modifier as the modifier changes position, orientation, dielectric properties, and/or configuration. The rate of repeating the correction procedure may depend on the rate at which the field modifier changes, and the accuracy demands put on the following process: if less accurate following is acceptable, a slower rate of repetitions may be used.

Potential advantages in using electrical measurements from a second device in order to guide its navigation and/or estimate its position include reducing or eliminating a need for imaging and/or position finding using exogenous measurement methods. For example, it may be possible to eliminate use of an esophagus-inserted (or other endoscopically positioned) ultrasound transducer and/or ionizing radiation imaging methods. This may in turn obviate a need for the use of general anesthesia and/or the need for an attending anaesthesiologist thereby reducing the overall cost of medical procedures. Patient recovery time may also be faster for procedures performed without the use of general anesthesia.

An aspect of some embodiments of the present disclosure relates to the electrical field-guided positioning of a second device within a body cavity, using electrical field mapping information generated from electrical field measurements by electrodes of a first device, wherein the second device is different in shape and/or electrical characteristics from electrodes of the first device.

For example, the electrodes of the first device may be relatively small—e.g., a millimeter or two in length and/or diameter.

The second device, in contrast, may be an implantable device or other medical implement comprising an electrically conductive portion of several millimeters, in each of one or more dimensions, having a surface which is exposed and/or configured to be exposed upon intralumenal deployment and/or operation of the second device. The electrically conductive portion of the second device is used as the electrode portion of the device. Exemplary sizes of the electrically conductive portion, in each of the one or more dimensions include sizes larger than the measurement electrodes of the first device by a factor of at least 2, 3, or 4, and/or at least 5 mm, at least 10 mm, 20 mm, or more. That conductive portion is wired by an insulated wire, in some embodiments, to form the electrode (electrode portion) used in making electrical field measurements.

The conductive portion may be used as an electrode, having a form provided primarily for a non-measurement function: for example, as a support, stent, plug, screw, anchor, needle, net, valve, frame, and/or other non-measurement function. In other words, whilst the conductive portion's primary function may be structural (i.e. not for measuring, but instead has a mechanical function), the conductive portion can have a secondary function as a measuring electrode. In some specific examples, the second device is a device (for example, Amplatzer Septal Occluder, Watchman LA appendage occluder, or a Cardioband anchor) which does not comprise electrodes, but comprises conductive structural portions that may be used as electrodes to measure parameters of electrical fields. In some embodiments, the deployment of the second device includes deployment of the conductive portion through a shape alteration, e.g., from a compact form (e.g., such as may be deliverable via a catheter) to an expanded form. In some embodiments, the exposed surface of the conductive portion is exposed by removal of to an insulating and/or partially insulating surface. For example, a metal stent coated with a fully or partially insulating coating may be exposed by having a portion of the coating removed in order to form an electrode (by exposing the conductive material that can function as an electrode).

In some embodiments, guidance of movement of the second device is performed using a rule which treats measurements made actually using the second device as if made by an electrode of the first device placed in some particular relationship to the second device. Herein, this is referred to as the equivalent first-device electrode position. For example, if the electrode portion of the second device is a linear conductor extending in a substantially linear (constant voltage gradient) electrical field, then the equivalent first-device electrode position is optionally at the geometrical center of the second device. For example, the equivalent first-device electrode position may be at the middle of the second device electrically conductive portion, and the rule may be “transform the measurements made by the second device to positions, using the rule generated for transforming readings of the first device to positions; and attribute the obtained position to the center of the electrode portion (the conductive portion) of the second device”.

Optionally, the equivalent first-device electrode position is fixed relative to the second device, as in the aforementioned example. In some embodiments, the equivalent first-device electrode position is dynamic relative to the second device. For example, insofar as the voltage gradients are known (from the map made using the first device), and the shape of the second device's conductive electrode portion is also known (e.g., based on its design and deployment stage), it can be determined using standard techniques what measurement would be made as the second device moves to different positions, and, accordingly, what the equivalent first-device electrode position is for each of those positions, even if not always the same relative to the position of the second device. For example, based on the gradients and the structure of the second device, an equivalent first-device electrode position may be determined as a position at which a point electrode should be standing in order to measure the same measurement as the electrode-portion of the second device measures; and the rule may be: “transform the measurements made by the second device to positions, using the rule generated for transforming readings of the first device to positions; and attribute the obtained position to the determined equivalent first-device electrode position”.

In some embodiments, calibrations (e.g., using any of the reference position types described herein above) take into account relative differences between first device and second device geometry. For example, an expanded second device potentially encounters a wall physically, even though its equivalent first-device position remains remote from the wall. The distance being known or estimated, a contact with the wall provides a potential calibration point for aligning, scaling, and/or confirming the alignment/scaling of the original first-device measurement map with measurements now made by the second device.

An aspect of some embodiments of the present disclosure relates to the electrical field-guided positioning of a second device within a body cavity, using electrical field mapping information generated from electrical field measurements by electrodes of a first device, wherein:

The second device is susceptible to assume a plurality of conditions which cause it to act as an electrode with different properties, and Among those conditions among which is a subset of conditions wherein it most closely approximates electrical field measurement characteristics of one or more electrodes of the first device.

For example, in some embodiments, the electrodes of the first device may be relatively small—e.g., a millimeter or two in length and/or diameter.

The second device, in contrast, may be an implantable device comprising an electrically conductive portion of several millimeters in one or more dimensions (e.g., in a range of 5-30 mm). That conductive portion is wired, in some embodiments, to form the electrode used in making electrical field measurements.

In some embodiments, the second device is delivered through and/or within an electrically insulating sheath. As the device leaves the sheath, in some embodiments, it gradually assumes different electrical properties affecting how it “sees” the electrical fields around it.

In some embodiments, navigation of the second device takes place while it is in a partially un-sheathed configuration which provides to it electrical field measurement characteristics similar to those of one or more of the electrodes of the first device. Herein, any such configuration is referred to as a “mimicking” configuration relative to one or more electrodes of the first device. For a second device in a mimicking configuration, the rule for applying electrical field mapping information obtained using electrodes of the first device is optionally as straightforward as a direct lookup. Optionally (for example if the mimicking configuration mimics imperfectly), scaling and/or offset corrections (if applied at all) are made relatively simple and direct by use of the mimicking configuration. The inventors have found that a useful degree of mimicry is optionally obtained, in some embodiments, by extruding a second device until electrical field measurements begin to be received at measurement value levels (e.g. voltage values) corresponding (e.g., within about ±20%) to levels of first device electrode measurements at about the same location—and then stopping. Optionally, magnitudes of measurement noise are used as an indicator. For example, upon reaching, during device extrusion, reaching an impedance low enough to make measurements with a root mean square noise amplitude similar (e.g., within ±20%) to the noise amplitude of first-device electrodes is used as a signal that a mimicking device configuration has been reached.

In some embodiments, the second device is reconfigurable during navigation between the mimicking configuration and another configuration. The other configuration may be, for example, a more-unsheathed, more-extended and/or more-deployed configuration, or any other configuration that causes more of the conductive portion of the second device to be exposed. Optionally, this is used to obtain a direct calibration of the non-mimicking configuration to a rule using the electrical field mapping information from the first-device measurements. For example, the second device, in a mimicking configuration, makes measurements at a first position in the body cavity, and a rule applied to determine its position. The second device is then converted to a non-mimicking configuration, resulting in a known change in position relative to the determined position. More measurements are made. This can be repeated at one or more additional positions. The non-mimicking configuration, in some embodiments, comprises exposure of more of the surface of the second device outside an insulating sheath such as a catheter sheath; for example, at least 2×, 3×, 4×, or more surface along a longitudinal axis of the sheath than is exposed by an electrode of the first device. In some embodiments, the non-mimicking configuration comprises a conformational change in a shape of the second device, for example, a radial expansion of part of the device (e.g., an expansion to either side of a longitudinal axis of the device). Optionally, the radial expansion increases a diameter of the device by at least 2×, 3×, 4×, or more. In some embodiments, the non-mimicking configuration comprises exposing a surface of the second device which is specially formed to perform a non-electrical function. For example, the second device optionally comprises a screw to thread, serrations, tool-receiving surface (e.g., a hexagonal recess), or another surface which is shaped to perform a mechanical function such as fastening, cutting, and/or engaging with a mating surface.

In some embodiments, mimicking/non-mimicking configuration pairs of measurements provide calibration indications which allow the first-device mapping data to be recalibrated to measurements by the second device in the non-mimicking configuration. In some embodiments, these measurements, optionally with suitable extrapolations and interpolations, are used stand-alone as a new set of position-mapped measurements, which indicate and/or guide further positioning of the second device.

In some embodiments, the second device is optionally itself used to make measurements of the types described in relation to the first device, e.g., a cloud of measurements which is used to generate a rule converts electrical field measurements into positions. In embodiments wherein the second device is not addressable as a plurality of (electrically mutually isolated) electrodes, this potentially limits methods by which the rule is generated, for example to a non-self-scaled method, optionally based on methods, principles, and/or systems for position estimation without self-scaling mentioned hereinabove.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Methods of Second Device Navigation

Second Device Navigation with Selected Operations Illustrated

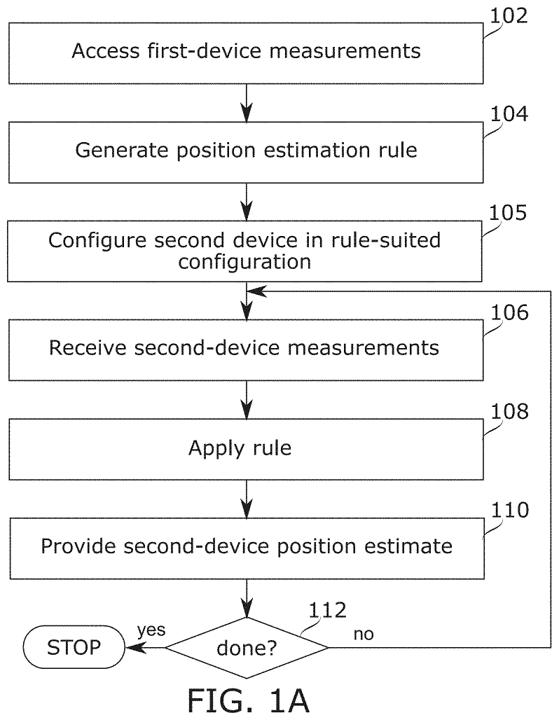

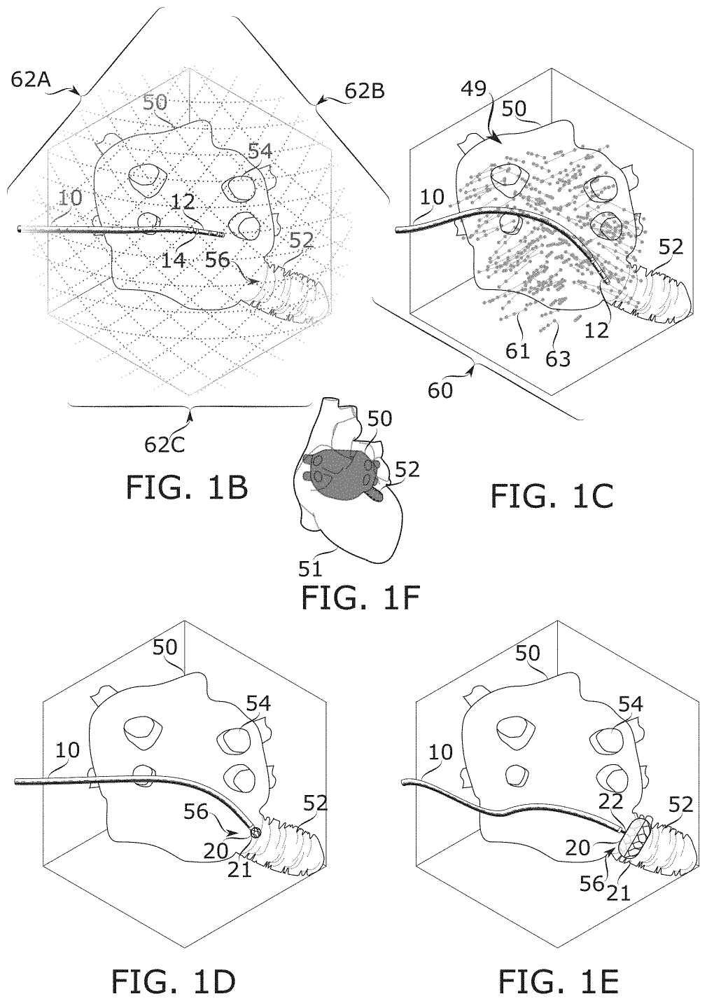

Reference is now made to FIG. 1A, which is a flowchart schematically illustrating a method of navigating a device within a body cavity 49 using electrical field measurements, according to some embodiments of the present disclosure. Further reference is made to FIGS. 1B-1E, which schematically illustrate an example of the method of FIG. 1A applied to device positioning (of a first device 12, and then a second device 20) in a body cavity 49 (in the illustrated example, a left atrium 50, shown in cross-section, of a heart 51), according to some embodiments of the present to disclosure. Further reference is made to FIG. 1F, which schematically illustrates an overview of left atrium 50 in relation to a heart 51, according to some embodiments of the present disclosure.

The flowchart of FIG. 1A begins; and at block 102, in some embodiments, electrical field measurements are accessed which were obtained from a first device 12 comprising a plurality of electrodes configured to measure electrical fields generated to extend through the body cavity 49. In some embodiments, the measurements accessed are voltage measurements indicative of electrical fields at the position that the measurements were made.

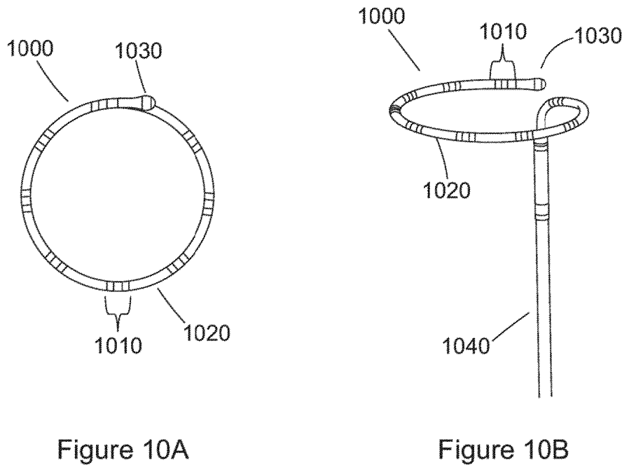

In some embodiments, first device 12 comprises an electrode catheter having two or more electrodes arranged at known distances from each other. A typical parameter that can be determined is impedance, which may itself be determined, e.g., from measurements of voltage made within one or more time-varying electrical fields. First device 12 is illustrated in FIG. 1A as a straight-probe electrode catheter. However, it should be understood that an electrode catheter of another configurations is used in some embodiments, e.g., a lasso configuration (loop-ended probe) as described in relation to FIGS. 10A and 10B.

In some embodiments, impedance is calculated from voltages/or and current measurements at electrodes of first device 12. In some embodiments, impedance is calculated from voltage measurements at electrodes of first device 12 and known current injected at electrodes of first device 12. In some embodiments, electrical measurement may include any dielectric parameter measured at one or more electrodes of first device 12. Electrical measurement may include voltage measurements, current measurements, impedance measurements and any combination thereof.

In some embodiments, measurements are accessed which were obtained from a first device 12 when the first device 12 was used for mapping and/or reconstructing the body cavity; e.g., to obtain measurements for a map and/or reconstruction used in an interventional procedure. Mapping and/or reconstructing the body cavity may be performed, for example, as described in International Patent Publication No. WO2018/130974 and/or in International Patent Publication No. WO2019/034944, the contents of which are included herein by reference in their entirety. In some embodiments, measurements are accessed which were obtained from a first device 12 when the first device 12 was used solely for obtaining such measurements, e.g., for calculating field mapping information to be used in navigation of the second device.

FIGS. 1B-1C illustrate a first device 12 (introduced, for example, through catheter sheath 10) comprising a plurality of electrodes 14 and used to map a region of a left atrium 50 of a heart 51, including at least an ostial portion 56 of a left atrial appendage (LAA) 52, and/or one or more pulmonary vein ostia 54. The darkened region of FIG. 1F represents the location of a left atrium 50 and LAA 52 relative to heart 51.

At block 104, in some embodiments, a rule for transforming electrical field measurements to positions is generated, using the electrical field measurements made by the first device 12. Herein, such a rule is also referred to as a position estimation rule.

In some embodiments, blocks 102 and 104 may be omitted, and replaced by accessing a position estimation rule. The position estimation rule may, for example, have been previously generated from measurements which were obtained from a first device 12 comprising a plurality of electrodes configured to measure electrical fields generated to extend through the body cavity 49. In other words, the position estimation rule may have been generated at a separate, earlier point in time by carrying out steps 102 and 104, but the disclosed methods may not necessarily explicitly involve these steps and may instead involve accessing the previously generated position estimation rule.

Electrical fields 62A, 62B, 62C (represented as a crossing grid of dotted lines in FIG. 1B) represent three electrical fields generated to extend through a body cavity 49 (including, in the example, left atrium 50). In some embodiments, electrical fields 62A, 62B, 62C are generated to extend through body cavity 49 by passing electrical currents between body surface electrodes at different electrical potentials. In some embodiments, body surface electrodes are provided on body surface patches, e.g., 6 body surface patches. In some embodiments, an additional ground patch may be used, e.g., a ground patch positioned on the patient's right leg. In some embodiments, the electrical fields are generated using intrabody electrodes (i.e. electrodes placed inside the body) instead of or in addition to surface electrodes.

A plurality of electrical fields are generated (e.g., between different electrode sets, and optionally differentiated by being generated at different times and/or frequencies). Each electrical field may be treated as defining a different coordinate of a coordinate system defined by the fields taken together. Measurement sets, each set measuring a plurality of electrical fields extending through an individual position within the body cavity 49, provide a distinctive “tag” for that position. Each such a “tag” may be considered the position of the electrode that took the measurement, expressed in coordinates of the field-generated coordinate system.

Generating electrical fields having mutually orthogonal voltage gradient components helps ensure that each position is associated with a different combination of electrical field measurements. However, the electrical field gradients are not necessarily orthogonal. While three voltage fields are illustrated in FIG. 1B, this is for purposes of illustration, and the number of voltage fields used may be larger or smaller.

The marking dots 63 used to represent measurement cloud 60 (FIG. 1C) represent different positions of first device 12 at which corresponding different sets of electrical field measurements are made. Sets of marking dots 63 joined by line segments 61 represent positions of simultaneously made measurements, with each dot 63 on a particular line segment 61 represents a position of a respective electrode 14 of first device 12, optionally each electrode along a line segment separated from each other electrode on the line by a respective known distance.

Nearby positions, in some embodiments, tend to be associated with correspondingly relatively similar sets of voltage measurements. However, relative positions of measurements 61 of measurement cloud 60 may not be initially known. For example, the distribution of electrical field gradients may include significant non-linearities with respect to their corresponding magnitudes and orientations in space. This potentially interferes with the use of electrical field measurements as a direct indication of position; e.g., isopotential lines of electrical fields bend through space, and may be closer or further apart depending on the distribution of structures with different dielectric properties through which the electrical fields extend.

However, position is potentially recoverable by using the measurement values themselves, along with additional information, to produce a reconstruction of the spatial distribution of the positions from which the fields have been measured. Herein, such a reconstruction is also referred to as a map.

In some embodiments, the rule for transforming electrical field measurements to positions optionally includes a rule for transforming a voltage measurements cloud (e.g., a group of measurements defined in a space with dimensions corresponding to measurements of different electrical fields and/or by different electrodes) to a position cloud (e.g., positions in a model of three-dimensional physical space associated with the measurements). In some embodiments, the rule may include a rule for transforming voltage measurements to positions within the body cavity.

In some embodiments, the position estimation rule generated in block 104 comprises use of such a map.

The map is optionally created using one or more of several available methods. Methods of converting electrical field measurements made by an electrode catheter to a map of positions at which those measurements were made are described, for example, in International Patent Publication No. WO2018/130974 and/or in International Patent Publication No. WO2019/034944, the contents of which are incorporated herein by reference in their entirety. In some embodiments, known distances between electrodes on an electrode catheter or other electrode probe are used to constrain the relative positions at which simultaneous measurements are made. The mutual distance constraint (indicated by the connecting lines 61) provides a kind of “ruler” that allows local scaling to be determined, in some embodiments, even when the change in voltage and/or impedance measured as a function of position is variable in magnitude and/or direction. Additionally or alternatively (for example), the problem of non-linearity is managed by computational modeling of the electrical fields, and/or by the conditions of generating the electrical fields: e.g., electrodes are optionally placed at sufficient distances and relative angles relative to the body cavity 49, so that significant portions of the electrical field change about linearly as a function of position offset along some direction.

The position estimation rule generated in block 104, in some embodiments, may be considered equivalent to application of a mapping between values of electrical field measurements made using the first device 12, and corresponding positions in space. Optionally, the rule includes additional features, such as modification for calibration with measurements made by a second device 20 having different electrical measuring characteristics than the electrodes of the first device 12. In some embodiments, the rule is supplemented by additional information; for example: constraints on where and/or when it is valid, and/or how it may be interpolated and/or extrapolated to positions which were not measured.

Optionally, the measurements from which a rule is generated are “over-determining”—that is, more measurements (e.g., of different electrical fields) are made at each position than are actually needed to uniquely identify positions. This potentially helps in reducing ambiguity and/or error in generating the rule. For example, there may be four or more electrical fields measured, even though the measurement values of only three crossing electrical fields may be sufficient to construct a rule which uniquely determines a position in space. A rule specifying spatial correspondences with just two, or even just one electrical field may be sufficient to determine position in a case where there are other constraints on position. For example, where position varies substantially along a single axis (e.g., because movement is constrained by advance out of or retraction into a catheter tube), position can potentially be determined from knowing the location of the axis in space (e.g., an orientation and a point of intersection), and a single electrical field measurement.

At block 105, in some embodiments, a second device 20 comprising an electrically conductive portion 21 (cf. FIGS. 1D and 1E) is configured to a configuration which is suited to application of the rule of block 104, when the second device 20 is placed within the body cavity 49 in which the measurements of the first device 12 were made. In some embodiments, no specific action is required for configuring the second device to a configuration suited to application of the rule, so block 105 may be omitted. For example, device 20 may have only a single configuration, which is suitable for the application of the rule. In another example, the device initial configuration is suited to application of the rule, so no active configuration is required. However, in other examples, an initial configuration of the second device is not suited to application of the rule, so the device 20 should be actively configured at step 105 as described below.

To be so-configured, the second device 20 is at least configured to act as an electrode within an electrical field measurement system. In particular, at least a conductive portion 21 of the second device 20 is itself electrically conductive (e.g., comprised of a metal or other low-electrical resistance material), and is furthermore connected to an electrical measuring device via a conductive member. “Configuring” the second device may therefore involve exposing at least a portion of the conductive portion as described in more detail below such that the conductive portion can “see” electrical fields and interact with them.

The conductive member, in some embodiments, is also a structural member to which the second device 20 is mechanically attached. The structural member may comprise, for example, a cable, tube, and/or strut which is operated to mechanically manipulate the second device 20; e.g., to extrude it from an electrically insulating catheter sheath. Where reference is made to a ‘structural member’, this is intended to refer to portions of the second device that are not primarily used for measurements. In other words, as described above, a structural member has a primary function that is mechanical, rather than to measure mechanical fields. A structural member is therefore not be designed to function as an electrode, but it is capable of functioning as a electrode as a secondary function.

The second device 20, in some embodiments, moreover comprises a deploying device; that is, a device which is delivered to the body cavity 49 in a first shape (for example, a shape suitable for movement along the lumen of a catheter tube), and deploys to a second shape (for example, a shape suitable for one or more functions of anchoring, blocking, moving, interconnecting, cutting, restraining, and/or affixing). In some embodiments, the second device 20 itself retains a substantially constant shape (e.g., a linear shape), but is optionally extruded more or less into the body cavity 49 from within a relatively insulating sheath. These changes in shape and/or extrusion distance also affect, in some embodiments, how measurements are made and/or how position-estimation rules are applied.

Two broad approaches by means of which a second device 20 and/or the position estimating system overall can be further configured to be suited to application of the rule of block 104 are (1) using and/or modifying the second device 20 so that it behaves more like electrodes of the first device 12 (for example by adjusting the length of the conductive portion of the second device that is exposed to act as an electrode), and (2) using calculations of how different physical and/or electrical characteristics of the second device 20 affect application of the rule of block 104. This is implemented, optionally, as modification of the rule of block 104 itself, and/or as further adjustments made upon application of the rule at block 108.

A third approach is to take measurements under special conditions which can be used to calibrate second device 20 measurements to first device 12 measurements. Any one, two, or three of these broad approaches are optionally used in some embodiments of the present disclosure. It is noted that in some embodiments, no calibration or rule modification is required, and the rule generated by the first device may be used for positioning the second device straightforwardly.

Electrodes of the first device 12 may each comprise, for example, a relatively small ring, e.g., of 1-2 mm diameter and 1-2 mm length (these measurements refer to the portions of the electrode electrically exposed to the environment of the body cavity 49, and exclude, for example, insulated wire conductors used to conduct current to a measurement device). The electrically conductive portion 21 of the second device 20, in contrast, may be larger (e.g., having at least one dimension in a range of 5-30 mm or larger) and/or more complex in shape, potentially resulting in different electrical field measurement properties.

In some embodiments, the second device 20 is made suited to application of the rule by being placed in a configuration selected to (in other words, by being configured to) mimic the electrical field measurement properties of an electrode of the first device 12. In a relatively simple example, the second device 20 is extruded from an insulating sheath to a small extent, e.g., so that only a portion of about the size of the electrodes of the first device 12 is electrically exposed to the environment of the body cavity 49 within which it is moving.

This “mimicking configuration” potentially helps to reduce complexity and/or increase accuracy of the rule for determining positions of the second device 20, as next explained. FIG. 1D shows an example of a second device 20 in a partially extended position which mimics an electrode 14 of first device 12. FIG. 1E shows the same device 20 in a fully deployed configuration, including an exposed portion of a connecting member 22, which is also a structural member used to advance and/or retract second device 20 relative to catheter sheath 10. The example of device 20 shown in FIG. 1D-1E is an LAA occlusion device, for which a goal of position estimation is to locate a tip of catheter sheath 10 at an ostium 56 of LAA 52 in preparation for deployment of device 20; and optionally another goal of position estimation is to monitor deployment of device 20.

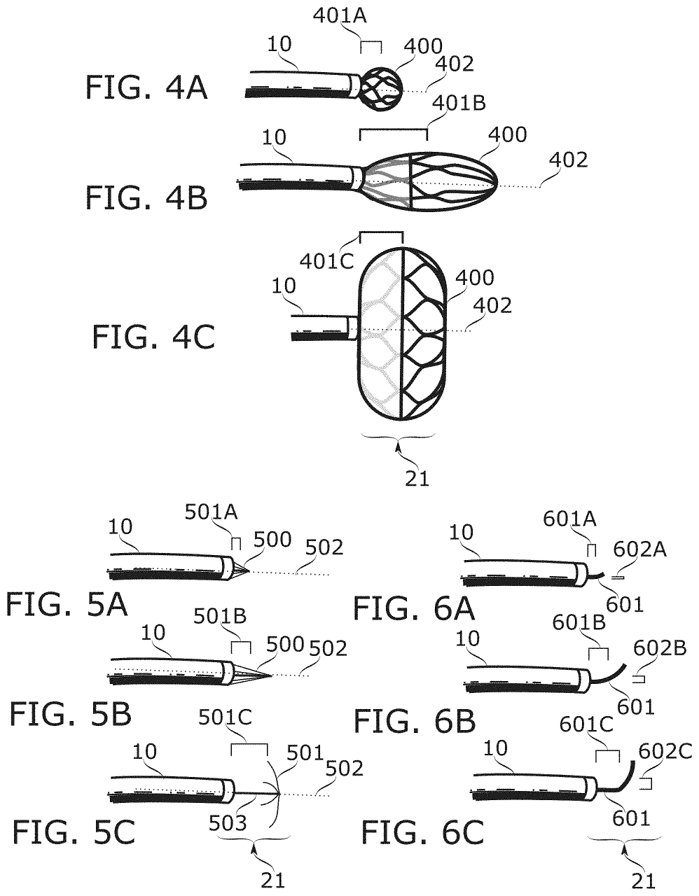

Examples of different devices and corresponding different configurations thereof are discussed, for example, in relation to FIGS. 4A-4C, 5A-5C, and 6A-6C.

When positions of the second device 20 are to be estimated while the second device 20 is not in a mimicking configuration, measurements made by the second device 20 are optionally subjected to further processing.

At block 106, in some embodiments, second-device measurements are received. At block 108, in some embodiments, the rule of block 104 is applied (optionally with additional corrections, for example, as next described; depending on whether the rule of block 104 is modified on the fly according to conditions of calibration and/or second-device configuration). At block 110, in some embodiments, the second-device position estimate is provided. In some embodiments, the second-device position estimate may be used for navigating the second device 20 within the body cavity, for example: during a medical procedure.

The operations of block 108-110 are optionally implemented by a rule as simple as a lookup performed on the map of first-device measurements to first-device position, using instead the second-device measurements of block 106. This is particularly well-suited to embodiments of the method wherein the second device 20 is operated in a mimicking configuration.

Alternatively, the rule includes additional corrections to adjust for differences between the second device 20 and the first device 12. In particular, corrections may be applied to account for the presence of field modifiers in the body cavity, as described above.

The electrically conductive portion 21 of the second device 20 is effectively at a single voltage potential (disregarding minor internal resistances and reactivity). More particularly, it is measured as having a single potential at any given time by the electrical measuring device to which it is connected. Accordingly, the second device 20, even when fully expanded, can be understood as having an equivalent first-device electrode position, given by applying the rule generated from first-device measurements to second-device measurements.

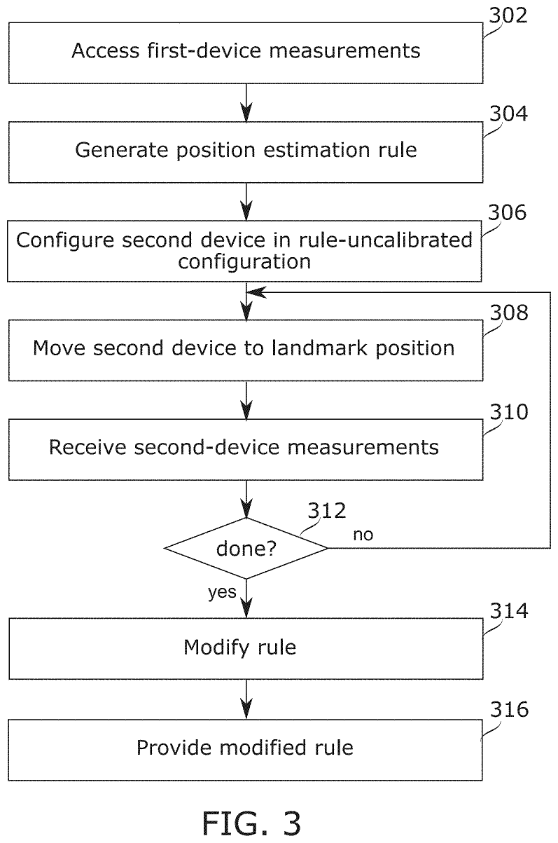

Second-device measurements of different electrical fields may, however, associate to different corresponding equivalent first-device electrode positions (even if measured simultaneously). This potentially complicates position determination, insofar as a set of simultaneous measurements made using the second device 20 may not, in fact, fully correspond to a measurement set made at any particular position of the first device 12. In some embodiments, this is overcome by calibration (methods of calibration are discussed herein, for example, in relation to FIG. 3) or other forms of correction, such as described above with reference to field modifiers. In some embodiments (with or without calibration), the rule of block 104 is adjusted to use an error-minimized lookup into the map of first-device measurements to first-device positions. For example, a measurement set made using the second device 20 is first associated to the closest available first-device measurement set (e.g., by a sum of absolute magnitudes of the difference, by least mean squares, and/or by another metric), and then to a position using the map.

The equivalent first-device electrode position of the second device 20 is likely to be within (or at least nearby) an envelope defined by the larger physical extent of the second device 20. Accordingly, in some embodiments, the position of the second device 20 is also defined as an envelope around the equivalent first-device electrode position (this can be implemented as a suitable modification to the rule of block 104). This allows estimation, e.g., of distances to contacts with walls of the tissue cavity, even though the equivalent first-device electrode position itself never reaches such a contact. For second devices 20 that undergo a conformational change during use (e.g., expansion during deployment): both the envelope, and its relationship to the equivalent first-device electrode position, are optionally varied as a function of shape. In some embodiments, these variations are made part of the rule of block 104. Different methods may be used to estimate a relationship between a second-device shape envelope and the equivalent first-device electrode position. In some simple implementations, a geometric center of the second device 20 (e.g., of its envelope, and/or of its center of mass) is assigned as the equivalent first-device electrode position. Optionally, interaction of the second device 20 with the electrical fields is electrically modeled, which can take into account, e.g., effects of electrical field gradient non-linearities, and/or influences of the second device 20 itself on the distribution of electrical field potentials. For example, such a model can take into account the presence of field modifiers in the cavity (such as conductive portions disposed on the tool) and the model can account for such field modifiers with a correction to the mapping rule or to the second device measurements. Conversely, the behavior of the equivalent first-device electrode position as a function of manipulation of the second device 20 is optionally used as an indication of second-device state, for example as described in relation to FIGS. 4A-4C, 5A-5C, and/or 6A-6C, herein.

From block 112, in some embodiments, further measurements, rule applications, and position estimates may be repeatedly performed as second device 20 navigates in body cavity 49 and/or as the second device is deployed. The iterative process may continue until no further estimate of the position of the second device 20 is required (for example when the position estimates converge within a predetermined error threshold). The second device 20 may be placed into different configurations in different loops, which can affect, for example, how the rule is applied at block 108.

Second Device Navigation in Two Configurations

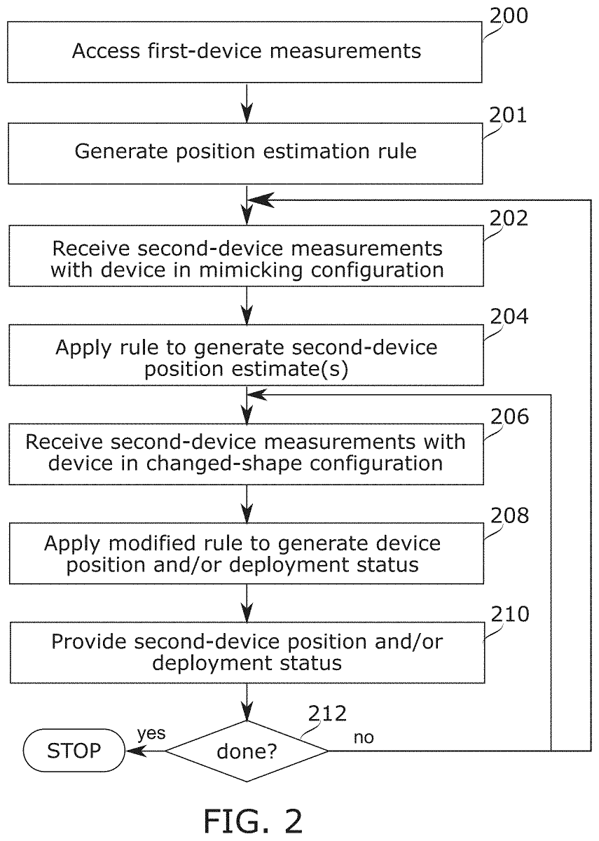

Reference is now made to FIG. 2, which is a flowchart schematically illustrating a method of positioning a second device 20 using a combination of mimicking configuration measurements and changed-shape configuration measurements, according to some embodiments of the present disclosure. In some embodiments, the method of FIG. 2 is more particular example of the method of FIG. 1A.

At block 200, in some embodiments, first-device measurements are accessed, and at block 201, a position estimation rule is generated. These blocks correspond, in some embodiments, to blocks 102 and 104 of FIG. 1A. In some embodiments, the method begins with accessing (and not necessarily with the generating of) a position estimation rule. In other words blocks 102 and 104 may be omitted, since the accessed position estimation rule may have been determined at an earlier, separate point in time. The position estimation rule may, for example, have been previously generated from measurements which were obtained from a first device 12 comprising a plurality of electrodes configured to measure electrical fields generated to extend through the body cavity 49.

At block 202, in some embodiments, second-device measurements are received, with the second device 20 in a mimicking configuration (for example, as described in relation to FIG. 1D and/or block 105 of FIG. 1A. At block 204, in some embodiments, the rule of block 201 is applied to generate a position estimate for the second device 20.

At block 206, in some embodiments, second-device measurements are received, with the second device 20 in a changed-shape configuration (for example, as described in relation to FIG. 1E and/or block 105 of FIG. 1A. At block 208, in some embodiments, a modified rule based on the rule of block 201 is applied to generate a position estimate for the second device 20. At block 210, the second-device position and/or deployment status is provided.

From block 212, in some embodiments, further measurements, rule applications, and position estimates may be performed until estimating of the position of the second device 20 ends or is no longer required.

In the first instance, operations of blocks 202 and 204 are distinguished from the operations of blocks 206 and 208 in that the second device 20 is in a differently-shaped configuration (mimicking configuration and changed-shape configuration).

In some embodiments, an additional distinguishing feature between blocks 202 and 204, and 206 and 208 is that the type of motion for which position estimations are generated is different. In some embodiments, movements during the position estimation in blocks 204 and 206 are free in three dimensions. This may be suitable, for example, for positioning to reach the deployment-ready position shown in FIG. 1D. Since second device 20 is in a mimicking configuration, in some embodiments, its position can be determined using a position-estimating map as if it carried an electrode like that of the first-device measurement device.

In some embodiments, position finding in blocks 206 and 208 is optionally performed while motion is constrained to operations which alter a deployment status of second device 20: for example, extrusion of second device 20 from catheter sheath 10 by advancing of connecting member 22, and/or actuation/deployment by other methods and/or control members. In some embodiments, second device 20 is elastically biased to self-expand or otherwise change shape as constraint from catheter sheath 10 is removed.

In cases where motion is limited to deployment movements, the position last estimated at block 204 is used, in some embodiments, as a base position. Subsequent position estimates are made as estimates of change from this position. For example, as second device 20 is extruded from catheter sheath 20, its equivalent first-device electrode position also advances, at least initially. With further expansion, there can also be a reversal of the direction of motion of the equivalent first-device electrode position, or another motion; for example, as described in relation to FIGS. 4A-4C, 5A-5C, and/or 6A-6C. Even though the overall shape and/or absolute position of second device 20 is not recorded by the measurements directly, it can optionally be calculated, with the understanding that movement of the equivalent first-device electrode position reflects a change in deployment status (shape) along a constrained range of possibilities. The movement can be understood in different ways, depending on the conformational changes undergone by the second device 20 as it deploys, for example as described in relation to FIGS. 4A-4C, 5A-5C, and/or 6A-6C, herein.

Additionally or alternatively, in some embodiments, interconversion between a mimicking configuration and a changed-shape configuration is performed several times, at different positions. Optionally, the positions of interconversion are used as calibration anchoring locations, allowing a rule which generates first-device positions from first-device measurements to be transformed (e.g., by interpolation between calibration anchoring locations) to generate second-device positions from second-device measurements, even when the second device 20 is in a configuration which does not mimic first-device electrodes. The calibration, in some embodiments, is between second-device measurements and first-device measurements; determining, e.g., adjustments in offset and/or scale of second-device measurements so that they can be used like first-device measurements in determining device positions.

### Second Device Measurement Calibration to First Device Measurement/Position Rules

Reference is now made to FIG. 3, which is a flowchart schematically illustrating a method of calibrating electrical field measurements made using a second device 20 within a body cavity 49 to electrical field measurements made using a first device 12 within the body cavity 49, according to some embodiments of the present disclosure.

The method of FIG. 3 shows a method of obtaining calibration anchoring locations, in which the position of the second device 20 is known from information other than its own electrical field measurements. This information is used to assign the electrical field measurements obtained using the second device 20 to a position, and through this assignment, determine what measurements the first device 12 would have made at the same position. From using several such calibration anchoring locations it can be determined how first-device electrical field measurements at that same position vary from the second-device electrical field measurements. Even two calibration anchoring positions are potentially sufficient e.g., to set a calibration scaling factor and/or offset along an axis of motion. As more calibration anchoring positions are obtained, the quality of the calibration is potentially improved.

The calibration, in some embodiments, is between second-device measurements and first-device measurements; determining, e.g., adjustments in offset and/or scale of second-device measurements so that they can be used like first-device measurements in determining device positions. Optionally, the calibration anchoring locations of the method of FIG. 3 are used along with calibration anchoring positions determined as described in relation to FIG. 2 to produce a calibration of second-device measurements to first-device measurements.

At block 302, in some embodiments, first-device measurements are accessed, and at block 304, a position estimation rule is generated. These blocks correspond, in some embodiments, to blocks 102 and 104 of FIG. 1A. In some embodiments, the method begins with accessing (and not necessarily with the generating of) a position estimation rule. In other words blocks 102 and 104 may be omitted, since the accessed position estimation rule may have been determined at an earlier, separate point in time. The position estimation rule may, for example, have been previously generated from measurements which were obtained from a first device 12 comprising a plurality of electrodes configured to measure electrical fields generated to extend through the body cavity 49.

At block 306, in some embodiments, a second device 20 is configured to a “rule-uncalibrated” configuration; that is, it is deployed into a shape which does not mimic an electrode of the first device. At block 308, the second device 20 is moved to a landmark position. This can be, for example, a position of catheter access into the body cavity 49 (e.g., a fossa ovalis or other point of transseptal penetration), and/or a position of farthest travel (e.g., a place at which advancement of the second device 20 is blocked by an encounter with a lumenal wall).

At block 310, in some embodiments, second-device measurements are received, which allows defining of a calibration anchoring position. From block 312, in some embodiments, the collection of calibration anchoring positions continues with repetition of block 308 and block 310 as necessary. Optionally, measurements at positions between well-defined landmarks (or offset from a single landmark in a known direction) are also collected for use as calibration anchoring positions, with their own positions being calculated according to the distance of advance along the path of travel. Once enough calibration anchoring positions have been collected, the method continues with block 314, wherein the position estimation rule of block 304 is modified, by use of the collected calibration information. At block 316, in some embodiments, the modified rule is provided. The modified rule becomes an optional basis for further position estimations, for example as described in relation to FIG. 1A, herein.

### Second Device Examples

Reference is now made to FIGS. 4A-4C, which schematically illustrate movement of an equivalent first-device electrode position of a cage-shaped second device as the second device deploys, according to some embodiments of the present disclosure.

In each of FIGS. 4A, 4B, and 4C, a second device 400 is shown in a different state of deployment (shape) as it is extruded from a catheter sheath 10. In some embodiments, second device 400 corresponds to the example of second device 20 illustrated, e.g., in FIGS. 1D-1E. Second device 400 corresponds, in some embodiments, to an LAA closing device. Such devices are used to close off the lumen of LAA from the general circulation. This potentially prevents blood clots which may form within the relatively static flow environment of the LAA from dislodging and entering the general circulation where they can create blockages and corresponding ischemia. In some embodiments, second device 400 is a self-expanding device; e.g., comprising nichrome struts compressed to a delivery configuration, and self-expanding once freed from confinement within catheter sheath 10.

In FIG. 4A, second device 400 is deployed by a small amount. Bracket 401A shows an estimated distance of the equivalent first-device electrode position from a distal tip of catheter sheath 10; equal to about half the overall distance of second device 400's deployment, or about where the center of gravity of the unsheathed and electrically conductive portion 21 of second device 400 is located. Local anisotropies in the electrical field environment may modify this estimate somewhat. In some embodiments, the modification is within a range small enough to be disregarded. Optionally, the modification is calculated and corrected for using the previously determined map of first-device electrical field measurements to positions.

For purposes of illustration, it may be presumed that the equivalent first-device electrode position is located along a central longitudinal axis 402 defined by the orientation of the distal tip of catheter sheath 10. However asymmetries of the second device 400 and/or the electrical field environment can potentially draw the equivalent first-device electrode position away from this central axis.

In FIG. 4B, deployment of second device 400 has proceeded further, and bracket 401B shows a corresponding increase in the distance of the equivalent first-device electrode position from the distal tip of the catheter sheath 10.

In FIG. 4C, second device 400 fully freed from within catheter sheath 10. As second device 400 finished expanding in radial directions, it also shortened again, causing its equivalent first-device electrode position to draw back toward the distal tip of catheter sheath 10. Bracket 401C indicates this shortened distance compared to bracket 401B.

This forward-then-backward movement is potentially characteristic of deployment for devices of this type, and is optionally used as a marker to help a physician track the stage of deployment. For example: before the reversal, the physician can be reasonably confident that the device is collapsed enough to allow easy repositioning; while after the reversal, the physical can be reasonably confident that the device is expanded enough to anchor. Reference is now made to FIGS. 5A-5C, which schematically illustrate movement of an equivalent first-device electrode position of an “umbrella”-shaped second device 500 as the second device deploys, according to some embodiments of the present disclosure.

The device of FIGS. 5A-5C is radially symmetric, like that of FIGS. 4A-4C, but with a different shape. The device shown has a central strut 503 with secondary struts 501 that expand to umbrella-like configuration. This could be, for example, an anchoring device (e.g., expanding after penetration of a lumenal wall by the central strut), and/or a device for deploying sensing nodes and/or ablation terminals. Regardless of function, second device 500 differs also from second device 400 during deployment in that it shows a different “final expansion” behavior. As secondary struts 501 are freed enough from catheter sheath 10 to expand, they tend to rapidly draw the equivalent first-device electrode position rapidly forward. Compare, for example, brackets 501A and 501B (wherein the equivalent first-device electrode position is about half the total advance of the second device 500) to bracket 501C, wherein the expanded struts have drawn the electrical center of the second device 500 much closer to its distal end.

Similarly to the situation in FIGS. 4A-4C, the equivalent first-device electrode position of second device 500 is expected to lie on or close to longitudinal axis 502. Any substantial deviation from this during deployment might indicate a problem, for example, an impediment to arm expansion, for which problem a physician may choose to take corrective action.

Reference is now made to FIGS. 6A-6C, which schematically illustrate movement of an equivalent first-device electrode position of a bent-linear second device 601 as the second device deploys, according to some embodiments of the present disclosure.

The device of FIGS. 6A-6C unsheathes into a bent-linear shape. Brackets 601A-601C show longitudinal advance of the equivalent first-device electrode position, substantially as described for the other devices, except that there is no position of sudden increase or reversal.

Brackets 602A-602C show increasing offset during unsheathing from a central longitudinal axis defined by the orientation of a distal tip of catheter sheath 10. This is due to a predefined bend in second device 601 which it assumes upon unsheathing. Such a bend might allow, for example, sideways access to a surface, and/or be a feature of a guidewire allowing selection of an off-axis aperture to advance into.

A physician monitoring the increase in radial offset optionally uses this to learn the direction of radial offset, and/or to help gauge if the device is expanding unimpeded, or if it is being impeded, for example, by contact with a lumenal wall instead of a targeted aperture.

### System for Second Device Position Estimation

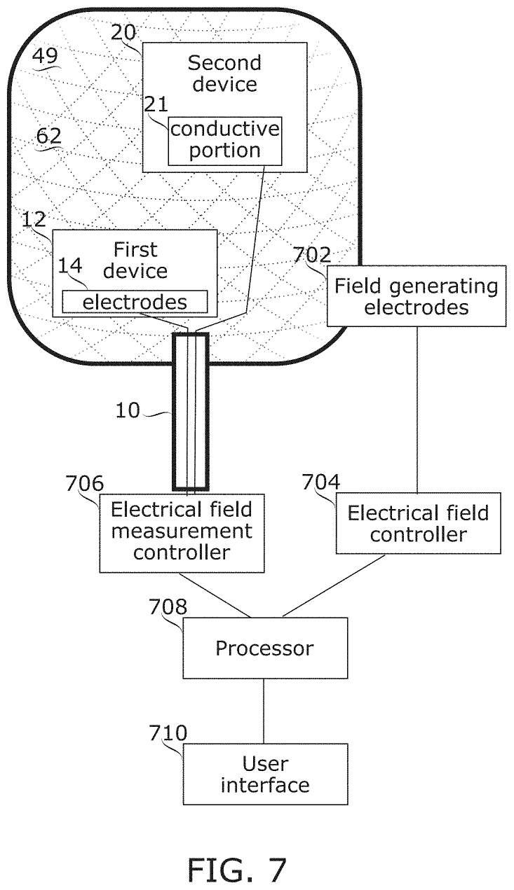

Reference is now made to FIG. 7, which schematically illustrates a system 700 for navigating a device within a body cavity 49 using electrical field measurements, according to some embodiments of the present disclosure. In some embodiments, the system of FIG. 7 is applicable, for example, to perform the method of any one or more of FIGS. 1A, 2, and/or 3.

In some embodiments, system 700 comprises one or more of:
Catheter sheath 10, for example as described in relation to FIGS. 1B-1E, 4A-4C, 5A-5C, and/or 6A-6C. In some embodiments, first device 12 and second device 20 are delivered through the same catheter sheath 10 (e.g., in alternation). In some embodiments, first device 12 and second device 20 are delivered through different catheter sheaths 10. In some embodiments, the first device is removed from the body cavity before the second device is inserted into the body cavity (either via the same or different catheter sheaths). In some embodiments, the second device may be inserted into the body cavity before the first device is removed, but the measurements of electrical fields from the second device are not made until the first device is removed from the body cavity. In some embodiments, an indication that the probe has been removed from the body cavity may be received before measurements are made using the second device.
First device 12—which may be, more particularly, an electrode catheter insertable to a body cavity 49—comprising a plurality of measurement electrodes 14, for example as described in relation to FIGS. 1B-1C. In some examples, the first device is a circular catheter as described in relation to FIGS. 10A and 10B.
Second device 20, which is a device insertable to a body cavity 49; for example as described in relation to FIGS. 1D-1E, 4A-4C (second device 400), 5A-5C (second device 500), and/or 6A-6C (second device 600), and/or FIGS. 9A-9B.
Electrical field generating electrodes 702, comprising a plurality of electrode which may be external (i.e., body surface) electrodes and/or electrodes insertable to a body to generate electrical fields (e.g., electrical fields 62, 62A, 62B, and/or 62C).
Electrical field controller 704, configurable to generate a plurality of electrical fields 62 (e.g., electrical fields 62A, 62B, 62C as illustrated in FIG. 1B) within a body cavity 49, wherein the electrical fields are optionally distinguished from each other by frequency, time of generation, and/or selection of electrode field generating electrodes 702 used to generate the electrical field. Optionally, a plurality of electrical field controllers combine to perform the functions of electrical field controller 704.
Electrical field measurement controller 706, configurable to receive electrical signals from an electrically conductive portion 21 of second device 20 and/or electrodes 14 of first device 12, optionally at the same time or alternately. In some embodiments, electrical field measurement controller comprises a voltmeter, ammeter, ohmmeter, impedance meter, or other electrical property measuring device. Optionally, a plurality of electrical field measurement controllers combine to perform the functions of electrical field measurement controller 706.
Processor 708, configured to receive and/or access electrical field measurements from the first device 12 and/or the second device 20, perform processing operations including position estimation rule generation and application, position estimation rule calibration, and/or position estimation rule modification. Optionally, processor 708 exerts control over electrical field controller 704 and/or electrical field measurement controller 706, e.g. to set and/or select parameters.
User interface 710, optionally used to display position estimation results and/or receive user input controlling operations of processor 708.
   Second Devices with a Plurality of Conductive Portions

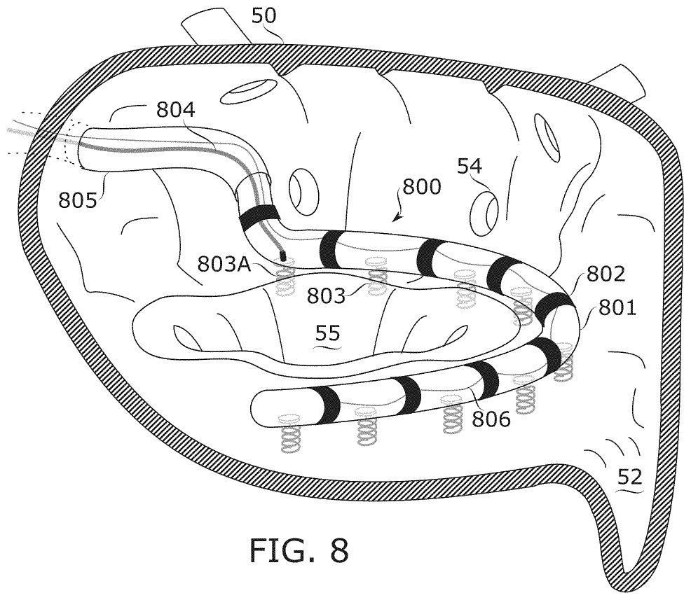

Reference is now made to FIG. 8, which schematically represents an implantable device 800 for modifying the circumference of a heart valve 55, and comprising a plurality of electrically conductive fasteners 803 (also referred to as “anchors”) used to secure implantable device 800 to the wall of a heart left atrium 50, according to some embodiments of the present disclosure. Heart left atrium 50 is shown in cross-section.

Implantable device 800, in some embodiments, comprises a flexible member 801, which is configured to be secured to tissue around the perimeter a heart valve 55 using fasteners 803 (optionally screws). Upon being secured, the device can be cinched, shortening flexible member 801 and drawing the perimeter tissue together. This potentially treats leakage/regurgitation through heart valve 55 (a heart mitral valve, in the example shown) by bringing leaflets of the valve closer together.

Implantable device 800 is shown during a late stage of implantation, with the flexible member 801 still partially within catheter sheath 805 (from which it has been partially extruded). Several fasteners 803 have already been inserted to tissue, and fastener 803A (also electrically conductive) is in the midst of being inserted. Insertion is performed, in some embodiments, by rotation of cable 804 to which fastener 803A is removably attached. Cable 804 is itself also electrically conductive, and in electrical contact with fastener 803A.

In some embodiments, each fastener 803 is brought separately from a proximal end of the catheter sheath 805 to be secured into place by mechanical operation of cable 804. In some embodiments, cable 804 is attached to an electrical field measuring device, thereby converting fastener 803 (e.g., fastener 803A) into an electrical field measuring electrode. In some embodiments, this allows fastener 803 to act as a second device, for example as described in relation to FIG. 1A. Optionally, manipulation of fastener 803 by cable 804 produces changing measurements during fastening. These measurements may indicate the position of fastener 803, for example as described in relation to FIG. 1A. Additionally or alternatively, information about the progress of implantation may be inferred from the measurements. For example, as more of fastener 803 embeds in tissue, it may acquire a higher impedance and/or a changed equivalent first-device electrode position. Optionally, this information is used to track progress of implantation.

Radio-opaque markers 802 are optionally comprised of a conductive material (e.g., a radio-opaque metal). Optionally one or more of radio-opaque markers 802 are connected to a conductive wire 806 which extends to a proximal side of catheter sheath 805, where it is optionally connected to an electrical field measuring device. Radio-opaque markers 802 are optionally connected to a single wire 806, or to a plurality of separately conducting wires 806. When separately wired, positions of individual radio-opaque markers are optionally determined according to their equivalent first-device electrode position. When wired to a same conductor in common, the relationship of the equivalent first-device electrode position to the positioning of the radio-opaque marker 802 is potentially offset, e.g., to near a geometrical “center of gravity” of the electrically joined-together radio-opaque markers 802, or another location.

Movements of the equivalent first-device electrode position during implantation may provide additional information about device status (which is optionally used to track progress of implantation). For example, as each new electrically joined-together radio-opaque marker 802 is exposed upon extrusion from catheter sheath 805, there is potentially a relatively rapid (e.g., step-function) jump in estimated position. As the radio-opaque marker 802 approaches the wall of the left atrium 50, there may be an additional rapid change in estimated position, e.g., insofar as field density lines may be more concentrated near the body cavity wall, so that the contribution of the radio-opaque maker 802 to the overall measurement changes more rapidly as a function of its change in position.

It is noted that one or more radio-opaque markers 802 wired as one or more electrically isolated electrodes and/or a fastener 803 electrically isolated from the radio-opaque markers 802, optionally together form an electrode system comprising a plurality of electrodes. By controlling distances of extrusion of flexible member 801 and fastener 803, the distances of these elements (considered as electrodes) is optionally controlled so that it is known, and this system, in some embodiments, then used as a plurality of electrodes for self-scaled mapping of the body cavity, for example, as described for a first device in the overview, and/or in relation to FIGS. 1A-1C, herein.

Also shown in FIG. 8 are left atrial appendage 52 and pulmonary vein roots 54.

### Example Second Device—Amplatzer Septal Occluder

As described above, the second device may be an implantable device and may have a conductive portion that has a primary function that is a non-measurement function (i.e. the conductive portion's primary function is structural). In some specific examples, the second device is an Amplatzer Septal Occluder as illustrated in FIGS. 9A and 9B.

FIG. 9A illustrates the Septal Occluder device 900 in relation to left heart atrium 920 and right heart atrium 910. The Septal Occluder is used to treat an atrial septal defect 930 (a hole in the wall 935 between the two atriums 910 and 920). The disclosed techniques can be used to determine a position of the device 900 to ensure that the device is passed through the septal defect 930 as shown in FIG. 9A.

FIG. 9B depicts a closer view of the device 900 as it is used to close up the septal defect 930. The device includes a first expanding portion 940 (which is also depicted in FIG. 9A) and a second expanding portion 950 (not shown in FIG. 9A). As shown in FIG. 9B, the first and second expanding portions are either side of the septal defect and clamp the device against the heart wall, thus sealing the septal defect up. FIG. 9B illustrates the device 900 after expansion of the first and second expanding portions, however it would be appreciated that the expanding portions are initially in a contracted configuration in order for the device to pass through guidewire 960 and through the septal defect 930, and are subsequently expanded to clamp the device in place over the septal defect.

The septal device includes one or more electrically conductive portions connected to an electrical field measurer via a conductive member such as a wire. The conductive portion can therefore function as an electrode, meaning that voltages, or other measurements indicative of electrical fields can be measured using the conductive portion. The conductive portion may be either or both of the expanding portions 940 and 950 themselves, which have a primary, structural (mechanical) function as described above, but are also capable of functioning as electrodes when connected to an electric field measurer by another conductive member.

### Example First Device—Circular Catheter

In some specific examples, the first device is a circular catheter, e.g., a Lasso® catheter, or a lasso-like catheter, for example as depicted in FIGS. 10A and 10B. FIG. 10A depicts a top-view of a Lasso® catheter whilst FIG. 10B depicts a side view of a Lasso® catheter. As can be seen from these drawings, the catheter 1000 comprises electrode pairs 1010 each comprising two electrodes, wherein the pairs are disposed on a loop portion 1020. The electrodes disposed on the loop portion may lie substantially flat in a plane defined by the loop. In other words, in some embodiments the loop portion 1020 is considered as a ring of electrodes that lie in a plane. This plane in which the electrodes lie and as defined by the loop is referred to herein as the lasso plane. It will be appreciated that other geometries that define a catheter plane can be used in place of the Lasso® catheter in these specific examples. In the specific embodiment depicted in FIGS. 10A and 10B, a tip electrode 1030 is also provided which is disposed at the end of the loop portion as illustrated, however the tip electrode may be omitted in some embodiments. In the example depicted in FIG. 10A, the catheter comprises 8 pairs of electrodes and a tip electrode, and in the example depicted of FIG. 10B, the catheter comprises 10 pairs of electrodes and a tip electrode. In some embodiments, the tip electrode may be omitted. In other examples (not depicted), the catheter may comprise 5 pairs of electrodes (i.e. 10 electrodes and a tip electrode), or any number of pairs up to 10 pairs plus a tip electrode. In examples, the diameter of the loop may be between 10 and 40 mm, and more specifically may be 12 mm, 15 mm, 20 mm, 25 mm, or 35 mm. In examples, the spacing between each pair 1010 of electrodes is between 4 mm and 11 mm, and more specifically may be 4 mm, 4.5 mm, 6 mm, 8 mm or 11 mm. The catheter may comprise a stem portion to which an end of the loop portion (the opposite end to which the tip electrode is disposed) is attached.

### General

It is expected that during the life of a patent maturing from this application many relevant implantable and/or endolumenally operated medical devices will be developed; the scope of the term implantable and/or endolumenally operated medical devices is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1. A method of guiding an electrically conductive medical implement inside a body cavity, the method comprising:
accessing a rule for transforming electrical field measurements to positions in the body cavity, wherein the rule is generated based on a first set of electrical field measurements of one or more electrical fields extending through the body cavity and which are measured using electrodes of a probe at various positions of the probe within the body cavity, the probe comprising a plurality of measuring electrodes;
receiving a second set of electrical field measurements of the one or more electrical fields measured via an electrically conductive medical implement within the body cavity, wherein the electrically conductive medical implement is a separate device to the probe; and
estimating a position of the electrically conductive medical implement when the second set of electrical field measurements was measured, using the second set of electrical field measurements and the rule.

2. The method of claim 1, wherein the electrically conductive medical implement comprises an electrically conductive portion having a surface of at least 10 mm in each of one or more dimensions, and is configured to expose the surface of the electrically conductive portion.

3. The method of claim 1, wherein the electrically conductive medical implement comprises an electrically conductive portion at least twice as big as each of the measuring electrodes of the probe in one or more dimensions, and is configured to expose a surface of the electrically conductive portion.

4. The method of claim 1, wherein the electrically conductive medical implement is configured to deploy, thereby exposing the electrically conductive portion to the electrical fields.

5. The method of claim 1, wherein the second set of electrical field measurements are received from the electrically conductive medical implement via a structural member of the medical implement, the structural member being electrically conductive and operable to move the electrically conducive medical implement within the body cavity.

6. The method of claim 4, wherein the second set of electrical field measurements is measured while the electrically conductive portion remains partially within a sheath from which it is configured to deploy.

7-9. (canceled)

10. The method of claim 1, wherein the rule for transforming electrical field measurements to positions transforms electrical field readings of the first set of electrical field measurements to positions of electrodes of the multi-electrode probe.

11. The method of claim 10, wherein estimating the position of the electrically conductive medical implement comprises applying the rule to measurements of the second set of electrical field measurements to produce a position result.

12. The method of claim 11, wherein the position result provides an estimated position for the electrically conductive medical implement.

13. (canceled)

14. The method of claim 11, comprising transforming the position result to an adjusted position result according to a model of the shape and size of the electrically conductive medical implement.

15-16. (canceled)

17. The method of claim 1, wherein the rule for transforming electrical field measurements to positions transforms electrical field measurements of the second set of electrical field measurements to positions corresponding to measurement positions of electrodes of the probe.

18-19. (canceled)

20. The method of claim 1, wherein the medical implement comprises a medical implant device configured to attach to and be left in the body.

21. The method of claim 1, wherein the medical implement is configured to expand in at least one of a longitudinal and a radial direction upon delivery to the body cavity, and the second set of electrical field measurements are made while the medical implement is expanded.

22. (canceled)

23. The method of claim 1, comprising receiving an indication that the probe has been moved from the body cavity before the second set of measurements was received.

24. The method of claim 1, wherein the rule for transforming electrical field measurements to positions is generated using inter-electrode distances of the multi-electrode probe.

25. The method of claim 1, wherein the estimating comprises use of a calibration of the second set of electrical field measurements to the rule using position information for the electrically conductive medical implement within the body cavity which is known separately from the second set of electrical field measurements.

26. The method of claim 25, wherein the position information known separately includes a known position of the electrically conductive medical implement at a septum of the heart.

27. The method of claim 25, wherein the position information known separately includes a position at which further advancing of the conductive medical implement is prevented by contact with a wall of the body cavity.

28. The method of claim 1, wherein the estimating comprises use of a calibration of the second set of electrical field measurements to the rule using one or more electrically measured characteristics of the environment of the electrically conductive medical implement.

29. The method of claim 28, wherein the one or more electrically measured characteristics of the environment comprise a change in measured impedance.

30. The method of claim 29, wherein the change in measured impedance is characteristic of proximity to a wall of the body cavity.

31. The method of claim 1, comprising moving the conductive medical implement, based on the estimating.

32. The method of claim 1, wherein the electrical field measurements are measurements of a parameter comprising at least one of the group consisting of current and voltage.

33-85. (canceled)
